(19) United States
(12) Patent Application Publication
(10) Pub. No.: US 2026/0331297 A1
(43) Pub. Date: Oct. 22, 2026

(54) SYSTEMS AND METHODS FOR GENERATING LATENT BIOLOGICAL REPRESENTATIONS AND HEALTH-RELATED PREDICTIONS FROM SEMANTICALLY MEANINGFUL INPUT GROUPS

(71) Applicant: Alden Scientific, Inc., Cambridge, MA (US)

(72) Inventors: James A. Heywood, Newton, MA (US); Dean E. Cerrato, Boston, MA (US); Kriston L. McGary, Flagstaff, AZ (US); Timothy E. Vaughan, Cambridge, MA (US)

(73) Assignee: Alden Scientific, Inc., Cambridge, MA (US)

(21) Appl. No.: 19/653,454

(22) Filed: Apr. 21, 2026

Related U.S. Application Data
(60) Provisional application No. 63/792,059, filed on Apr. 21, 2025.

(51) Int. Cl. G16H 50/30; G16H 10/60

(52) U.S. Cl. CPC G16H 50/30; G16H 10/60

(57) ABSTRACT

Systems and methods obtain health-related data for a subject, the health-related data being organized into a plurality of semantically meaningful input groups. Each input group is processed through a respective group-specific transformation pipeline relative to a common internal semantic basis to generate a corresponding group representation comprising a group-specific deviation. A respective adaptive weighting factor scales each group-specific deviation in a common slot-based semantic space, and the group-specific deviations are fused with the common internal semantic basis to generate an integrated representation aligned along common conceptual axes. The integrated representation is input into a machine-learning model to generate a latent biological representation of a biological state of the subject. The latent biological representation is used to generate at least one reconstruction output, including a denoised reconstruction, and at least one health-related prediction.

### CROSS REFERENCE TO RELATED APPLICATION(S)

This application claims the benefit of and priority to U.S. Provisional Application No. 63/792,059, filed Apr. 21, 2025, the contents of which is incorporated herein by reference in its entirety.

### FIELD OF TECHNOLOGY

Systems and methods herein related to technical fields of machine learning-based biological predictions health-related data and, more particularly, to machine-learning systems and computer-implemented methods for processing a plurality of semantically meaningful input groups relative to a common internal semantic basis in a common slot-based semantic space to generate a latent biological representation for reconstruction output and health-related prediction, such as using biological measurement(s) and correction of noise thereof using covariates.

### BACKGROUND OF TECHNOLOGY

Omics is the collective characterization and quantification of sets of biological molecules and the investigation of how those molecules translate into the structure, function, and dynamics of an organism or group of organisms. Examples of fields of omics include genomics, proteomics, metabolomics, metagenomics, phenomics, transcriptomics, among others.

In omics, typically, advanced systems capable of measuring microscopic molecules are needed in order to measure and track the behaviors of the molecules in organisms. Such systems can be subject to various sources of error which can affect downstream analysis. Thus, error correction techniques can be used to increase the fidelity of measurements to actuality.

### SUMMARY

In some aspects, the techniques described herein relate to a method including: obtaining, by the at least one processor, training biological measurement data from at least one multi-omics data source, the training biological measurement data including a plurality of training biological measurements for a plurality of biological measurements of a plurality of training subjects; determining, by the at least one processor, for a particular biological measurement of the plurality of biological measurements in the training biological measurement data, cross-biological measurement correlations between: at least one particular training biological measurement of the particular biological measurement and each covariate biological measurement of a plurality of covariate biological measurements associated with each covariate biological measurement, each covariate biological measurement being associated with a different measurement type relative to the training biological measurement data; updating, by the at least one processor, training for a biological measurement noise model to output at least one denoised biological measurement value based at least in part on cross-biological measurement correlations for the particular biological measurement, wherein the training includes: inputting each covariate biological measurement associated with each covariate biological measurement; outputting the at least one de-noised biological measurement value; and updating the biological measurement noise model based at least in part on an error between the at least one de-noised biological measurement value and the at least one particular training biological measurement of the particular biological measurement; utilizing, by the at least one processor, the biological measurement noise model to predict new de-noised biological measurement values for the particular biological measurement in new samples based at least in part on each new covariate biological measurement associated with each new covariate biological measurement of the plurality of covariate biological measurements in the new samples; and correcting, by the at least one processor, the particular biological measurement data for the new samples based at least in part on the at least one de-noised biological measurement value.

In one embodiment, the disclosure includes a computer-implemented method comprising obtaining health-related data for a subject, the health-related data being organized into a plurality of semantically meaningful input groups; processing each input group through a respective group-specific transformation pipeline relative to a common internal semantic basis to generate a corresponding group representation comprising a group-specific deviation from the common internal semantic basis; determining, for each input group, a respective adaptive weighting factor for scaling a contribution of a corresponding group-specific deviation in a common slot-based semantic space defined by the common internal semantic basis; fusing, within the common slot-based semantic space, the group-specific deviations according to the respective adaptive weighting factors with the common internal semantic basis to generate an integrated representation in which the plurality of semantically meaningful input groups are aligned along common conceptual axes; inputting the integrated representation into a machine-learning model to generate a latent biological representation of a biological state of the subject; and using the latent biological representation to generate at least one reconstruction output and at least one health-related prediction.

In some embodiments, the plurality of semantically meaningful input groups may comprise at least a proteomics group, a clinical covariates group, and a metabolomics group.

In some embodiments, the common internal semantic basis may comprise a plurality of learned shared concept anchors defining the common slot-based semantic space.

In some embodiments, processing each input group through the respective group-specific transformation pipeline may comprise converting features of the input group into an internal token representation and refining the internal token representation using intra-group contextual modeling before said fusing.

In some embodiments, processing each input group through the respective group-specific transformation pipeline may comprise interacting the input group with the common internal semantic basis through attention-based pooling to generate the corresponding group-specific deviation.

In some embodiments, the respective adaptive weighting factor for each input group may comprise a learned gate representing at least one of a confidence factor, a salience factor, or an influence factor for the corresponding input group.

In some embodiments, the computer-implemented method may further comprise refining the integrated representation with a shared encoder before said inputting the integrated representation into the machine-learning model.

In some embodiments, the at least one reconstruction output may comprise a denoised reconstruction generated by reconstructing entries intentionally corrupted or masked during training.

In some embodiments, the at least one health-related prediction may comprise at least one of a clinical outcome prediction, a disease-state classification, a continuous phenotype estimate, a survival-related prediction, or a treatment-response prediction.

In some embodiments, the computer-implemented method may further comprise performing an ablation-based analysis by removing or attenuating one of the plurality of semantically meaningful input groups and measuring an effect on the at least one health-related prediction.

In another embodiment, the disclosure includes a system comprising at least one processor and a memory storing instructions that, when executed by the at least one processor, cause the system to obtain health-related data for a subject, the health-related data being organized into a plurality of semantically meaningful input groups; process each input group through a respective group-specific transformation pipeline relative to a common internal semantic basis to generate a corresponding group representation comprising a group-specific deviation from the common internal semantic basis; determine, for each input group, a respective adaptive weighting factor for scaling a contribution of a corresponding group-specific deviation in a common slot-based semantic space defined by the common internal semantic basis; fuse, within the common slot-based semantic space, the group-specific deviations according to the respective adaptive weighting factors with the common internal semantic basis to generate an integrated representation in which the plurality of semantically meaningful input groups are aligned along common conceptual axes; input the integrated representation into a machine-learning model to generate a latent biological representation of a biological state of the subject; and use the latent biological representation to generate at least one reconstruction output and at least one health-related prediction.

In some embodiments, the plurality of semantically meaningful input groups may comprise at least a proteomics group, a clinical covariates group, and a metabolomics group.

In some embodiments, the common internal semantic basis may comprise a plurality of learned shared concept anchors defining the common slot-based semantic space.

In some embodiments, processing each input group through the respective group-specific transformation pipeline may comprise converting features of the input group into an internal token representation and refining the internal token representation using intra-group contextual modeling before said fusing.

In some embodiments, processing each input group through the respective group-specific transformation pipeline may comprise interacting the input group with the common internal semantic basis through attention-based pooling to generate the corresponding group-specific deviation.

In some embodiments, the respective adaptive weighting factor for each input group may comprise a learned gate representing at least one of a confidence factor, a salience factor, or an influence factor for the corresponding input group.

In some embodiments, the instructions, when executed by the at least one processor, may further cause the system to refine the integrated representation with a shared encoder before said inputting the integrated representation into the machine-learning model.

In some embodiments, the at least one reconstruction output may comprise a denoised reconstruction generated by reconstructing entries intentionally corrupted or masked during training.

In some embodiments, the at least one health-related prediction may comprise at least one of a clinical outcome prediction, a disease-state classification, a continuous phenotype estimate, a survival-related prediction, or a treatment-response prediction.

In some embodiments, the instructions, when executed by the at least one processor, may further cause the system to perform an ablation-based analysis by removing or attenuating one of the plurality of semantically meaningful input groups and measuring an effect on the at least one health-related prediction.

In some embodiments, the term “biological measurement” may refer to measurement of one or more traits, characteristics, abundances, presence, concentration, effects, or other aspect or any combination thereof pertaining to a biological sample.

In some embodiments, the term “sample” or “biological sample” may refer to biological materials obtained from living or deceased subjects, and may include, without limitation, biofluids, tissue, cells, ribonucleic acid, deoxyribonucleic acid (DNA), protein lysate, cell-free DNA (cfDNA), circulating tumor DNA (ctDNA), among others or any combination thereof.

In some embodiments, “biofluid” may include, without limitation, blood, bile, bone Marrow Aspirate, breast milk, Cerebral spinal Fluid (CSF), plasma, saliva, serum, sputum, stool, swabs (oral, nasal, vaginal fluids), synovial fluid, urine, etc.

In some embodiments, “tissue” may include, without limitation, samples collected from a specific organ of the subject, such as, e.g., FFPE blocks, fixed tissue, fresh tissue, frozen tissue, slides, tissue microarrays (TMA), etc.

In some embodiments, the term “measurement type” may refer to one or more categories of measurement techniques, modalities, etc. for measuring a particular biological measurement.

In some embodiments, the term “in-band” refers to a particular measurement being of a same measurement type relative to another measurement.

In some embodiments, the term “out-of-band” refers to a particular measurement being of a different measurement type relative to another measurement.

In some embodiments, the term “protein measurement” may refer to a measured level, quantity, amount or other measurement of an associated protein present in a corresponding sample.

In some embodiments, the term “panel” may refer to a set of proteins grouped according to function, such as functions created based on, e.g., predefined, configurable, derived, or other technique(s).

In some embodiments, the term “block” and/or “block data set” may refer to a set of proteomic measurements that are, or have been, obtained according to one or more common or shared experimental conditions, such as a common reagent batch, assay plate, sample processing protocol, or other condition or any combination thereof. A block may refer toa single experiment (e.g., a batch from an assay plate), a data set from the same reagent lot, a computational unit of measurement grouping, among other groupings based on experimental conditions.

In some embodiments, the term “in-block” refers to measurements obtained within a given block, and may refer to measurements of the same type (e.g., protein abundance, immunoassays, blood platelet counts, nucleotide measurements, etc.), or may be any measurement from within the block.

In some embodiments, the term “out-of-block” refers to measurements obtained from a block different from that of a given measurement, and may refer to measurements of the same type (e.g., protein abundance, immunoassays, blood platelet counts, nucleotide measurements, etc.), or may be any measurement from outside of the block of the given measurement.

In some embodiments, the term “block control” may refer to error correction, noise correction, or other pre-processing applied to a block and/or block data set.

In some embodiments, the term “assay” may refer to an investigative (e.g., analytic) procedure for qualitatively assessing or quantitatively measuring the presence, amount, or functional activity of a target entity, such as a protein measurement, block control value, or other analyte, measurand, or target, among others or any combination thereof.

In some embodiments, the term “response variable,” “dependent variable,” “response measurement,” “dependent measurement,” “response protein,” “dependent protein,” or the like, may refer to a variable of interest to a particular study or analysis, such as the protein measurement value(s) for a particular measurement(s) of interest 122.

In some embodiments, the term “covariate” may refer to a variable that affects a response, or dependent, variable, but not of the variable of interest, such as a biological measurement that influences, is influence by, or is otherwise correlated with the protein measurement value(s) for a particular measurement(s) of interest 122.

In some embodiments, the term “correlate” and/or “correlated” and/or “correlation” may refer to a degree of association between measurements, the degree being statistically derived, established by criteria based on prior knowledge and/or reliability of measurement, among other forms of assessing association between measurements or any combination thereof.

In some embodiments, the term “noise” may refer to a systematic tendency in which the methods used to measure and/or gather data and generate statistics present an inaccurate, skewed or otherwise incorrect representation of actuality, such as a systematic offset to protein measurements.

### BRIEF DESCRIPTION OF THE DRAWINGS

Various embodiments of the present disclosure can be further explained with reference to the attached drawings, wherein like structures are referred to by like numerals throughout the several views. The drawings shown are not necessarily to scale, with emphasis instead generally being placed upon illustrating the principles of the present disclosure. Therefore, specific structural and functional details disclosed herein are not to be interpreted as limiting, but merely as a representative basis for teaching one skilled in the art to variously employ one or more illustrative embodiments.

FIG. 1A, FIG. 1B, FIG. 1C, and FIG. 1D depict block diagrams that illustrate the use of a biological prediction model 130 in determining and correcting for noise in protein measurement using covariate measurements, in accordance with one or more embodiments of the present disclosure.

FIG. 2 is a schematic block diagram illustrating a system for automated error correction of biological measurements using covariate biological measurements in accordance with one or more embodiments of the present disclosure.

FIG. 3 illustrates a schematic flow chart diagram of a method for determining correlations between measurement(s) and covariate measurements different from the measurement(s) of interest in accordance with one or more embodiments of the present disclosure.

FIG. 4A depicts a block diagram illustrating the interaction between biological measurements, covariate biological measurements, and a prediction model with one or more optimization functions in accordance with one or more embodiments of the present disclosure.

FIG. 4B depicts a block diagram illustrating the creation of training data for training the biological prediction model using simulated noise of biological measurements in accordance with one or more embodiments of the present disclosure.

FIG. 5A depicts a flowchart illustrating a grouped multimodal architecture for generating a latent biological representation from proteomics, clinical covariates, and metabolomics.

FIG. 5B depicts a block diagram illustrating a biological prediction model architecture including grouped measurements, fusion, a latent biological representation layer, reconstruction layer(s), and prediction layer(s) in accordance with one or more embodiments of the present disclosure.

FIG. 5C depicts a block diagram of an exemplary biological prediction model utilizing multi-layered deep neural network machine learning to output both de-noised biological measurements and health-related predictions in accordance with one or more embodiments of the present disclosure.

FIG. 6 is a block diagram illustrating a system for reducing noise in biological molecule measurement systems in accordance with one or more embodiments of the present disclosure.

### DETAILED DESCRIPTION

Various detailed embodiments of the present disclosure, taken in conjunction with the accompanying FIGs., are disclosed herein; however, it is to be understood that the disclosed embodiments are merely illustrative. In addition, each of the examples given in connection with the various embodiments of the present disclosure is intended to be illustrative, and not restrictive.

Throughout the specification, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments may be readily combined, without departing from the scope or spirit of the present disclosure.

In addition, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

As used herein, the terms “and” and “or” may be used interchangeably to refer to a set of items in both the conjunctive and disjunctive in order to encompass the full description of combinations and alternatives of the items. By way of example, a set of items may be listed with the disjunctive “or”, or with the conjunction “and.” In either case, the set is to be interpreted as meaning each of the items singularly as alternatives, as well as any combination of the listed items.

In the field of biological measurement, accurate data collection and analysis are important for understanding the complex interactions and functions of a particular biological measurement within organisms. However, the measurement can be fraught with challenges, including noise introduced during the data collection process. These inaccuracies can significantly impact the reliability of downstream analyses and the application of predictive models across different data sets. For instance, when attempting to apply models trained on one cohort to another, discrepancies in measurement values can lead to inconsistent and unreliable results.

Existing methods for noise correction and/or reduction of biological measurement data often struggle to address these issues effectively. Indeed, traditional methods for noise correction or normalization for biological measurement data often rely on direct numerical comparisons or simple statistical adjustments. These approaches can be inadequate, as they do not account for complex inter-measurement relationships, or the specific noise introduced in a particular sample and/or block.

These problems are relevant to any field of omics, where the detection and correction of noise may pose challenges in genomics, proteomics, metabolomics, metagenomics, phenomics, transcriptomics, among others or any combination, and indeed in other biological measurements including biomarkers and physiological and anatomical measurements, among others or any combination thereof. Thus, while, purely for illustration and ease of understanding, the following may make reference to examples including proteomics, inventors have appreciated that the techniques herein may be applied in any field of omics, and indeed in other biological measurement paradigms.

For example, biological measurement data, including multi-omics data, can be affected by structured noise introduced by measurement platforms, assays, blocks, samples, preprocessing operations, or other technical factors. Such noise can mimic underlying biology, distort relative measurement values, and reduce the reliability of downstream analysis. In particular, when predictive models are trained on one cohort or data source and applied to another, differences in measurement noise can impair transferability and lead to inconsistent outputs. Conventional correction approaches that rely primarily on direct numerical normalization or limited statistical adjustment may be unable to capture complex relationships among different biological measurements or to account for noise patterns that vary across samples, blocks, or measurement types.

To address these issues, the disclosed techniques may use a machine-learning architecture that infers an expected biological signal from correlated information distributed across multiple biological and clinical measurements. In some embodiments, measurements of interest and associated in-band or out-of-band covariates, including measurements from different omics modalities or other semantically meaningful data groups, are processed through corresponding pathways and mapped into a shared internal representational basis to form a latent biological representation. The architecture may leverage cross-biological-measurement correlations to reconstruct or predict de-noised measurement values, and may further use the same latent representation to support reconstruction of one or more data groups and generation of phenotypic, diagnostic, or risk-related predictions. In some embodiments, the relative influence of respective data groups may be adaptively modulated, and the architecture may be trained to remain effective when one or more groups are unavailable, thereby providing a flexible framework for noise reduction, robust multimodal integration, and downstream biological prediction.

In some example embodiments, the techniques herein can provide innovative approaches to data measurement denoising within different proteomics data sets, specifically addressing the challenge of measurement noise in multi-omics measurement data to provide a flexible, multi-stage noise reduction approach using in-band and/or out-of-band data signals, preserving biological signals. Such techniques can leverage cross-omic (e.g., cross protein, or cross gene, etc.) correlations to predict measurement values, thereby enabling the correction of noise introduced by the particular multi-omics platform, assay, block, sample, shared block controls, among others or any combination thereof. The approach involves training a biological prediction model using in-band and out-of-band measurements to predict and adjust protein values, which enhances the accuracy of predictive models when applied to different cohorts, such as the UKB and Aurora cohorts. By leveraging covariate out-of-band and in-band measurements, this method provides a more reliable framework for applying predictive models across diverse data sets, thus overcoming a significant impediment in the field of multi-omics data analysis.

In some examples, the measurement or measurement type may be protein abundances collected from a proteomic platform. The proteomic data sets may be obtained from one or more proteomics measurement systems, such as immunoassays, antibody-free protein detection (e.g., including Edman degradation, mass spectrometry-based techniques, etc.), separation-based techniques, mass spectrometric immunoassays (MSIA), Stable Isotope Standard Capture with Anti-Peptide Antibodies (SISCAPA), Fluorescence two-dimensional differential gel electrophoresis (2-D DIGE), affinity proteomics, protein detection via bioorthogonal chemistry, among others or any combination thereof.

In illustrative examples of embodiments herein, the data measurement denoising techniques may include processing noisy protein measurement data to extract true biological signals, mitigating the impact of structured noise and technical errors that obscure relative protein abundances in blood plasma. Uncorrected noise compromises the accuracy of predictive disease models. Thus, the proteomics platform can be improved by incorporating an approach to isolate the biological signal and enhance data reliability.

Indeed, protein measurement datasets can often contain structured noise that can mimic biological signals, leading to misrepresentation of true protein abundances. This noise impacts both direct interpretation and the predictive quality of machine learning (ML) models built using such data. The challenge is to develop an ML-based method that: extracts biological signals from noisy and/or multimodal data, filters out extraneous noise to reconstruct a normalized representation of data, enhances the accuracy of disease risk prediction models by leveraging de-noised protein data, and produces a compact, biologically relevant latent representation for downstream modeling and analysis.

According to embodiments herein, such denoising and processing can be effectuated with a deep neural network (DNN) architecture that concurrently reconstructs de-noised data from noisy measurements and predicts disease risk for multiple conditions. To do so, the DNN architecture can process multiple input channels, including multi-modal measurement data (e.g., measured values for thousands of proteins, clinical trait and lab measurements), and/or covariate data. Using such inputs, the DNN architecture can output predictions across multiple output channels concurrently, including: de-noised protein values (e.g., corrected representations of the input protein data), de-noised covariate values (e.g., reconstructed clinical lab values), numerical phenotypic traits (e.g., regression outputs predicting continuous health indicators), and phenotypic binary outcomes (e.g., classification outputs estimating the probability of future diagnoses), among others or any combination thereof.

In one embodiment, the system for obtaining and processing training multi-omics measurements data can be implemented using a distributed computing architecture, where multiple processors are employed to handle large datasets from various proteomics data sources. This setup allows for parallel processing of training multi-omics measurements, enhancing the efficiency and speed of data analysis. In another embodiment, the system may utilize a cloud-based platform to store and process the training multi-omics measurements data, providing scalability and remote accessibility for users. The cloud-based system can dynamically allocate resources based on the computational demands of the multi-omics measurements error model training and prediction tasks. Additionally, the system could incorporate machine learning algorithms that are specifically tailored to handle the distinct characteristics of proteomics data, such as high dimensionality and noise. These algorithms can be designed to automatically adjust their parameters based on the cross-multi-omics measurements correlations identified during the training phase, thereby improving the accuracy of the predicted multi-omics measurements measurement values. Furthermore, the system might include a user interface that allows researchers to visualize the cross-multi-omics measurements correlations and the impact of the in-band adjustments on the multi-omics measurements data, facilitating a deeper understanding of the measurement optimization process. This interface could be customizable, enabling users to select specific multi-omics measurements or blocks of interest for detailed analysis.

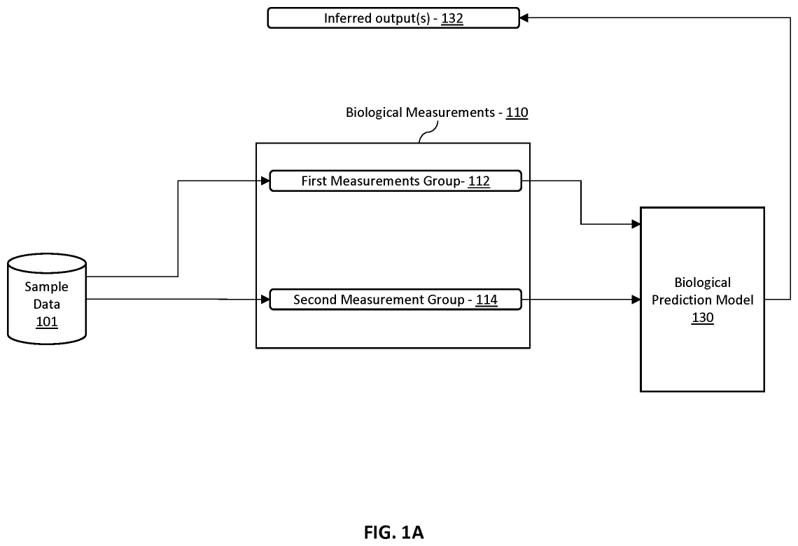

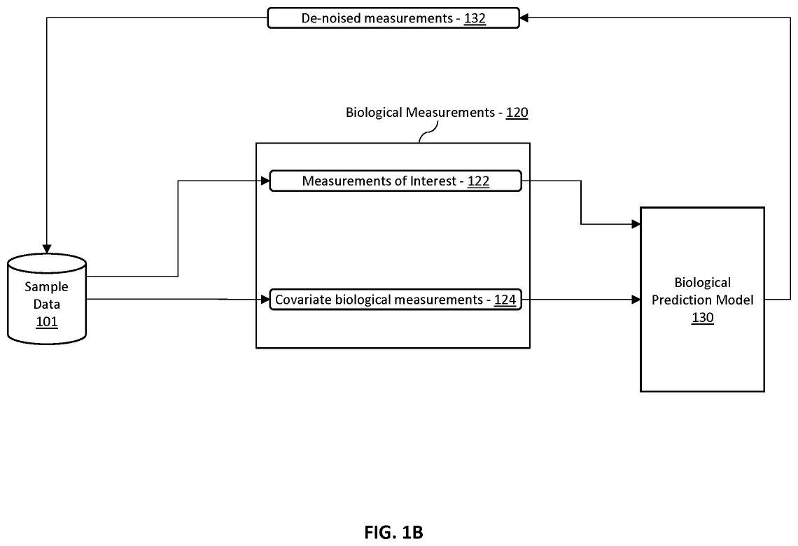

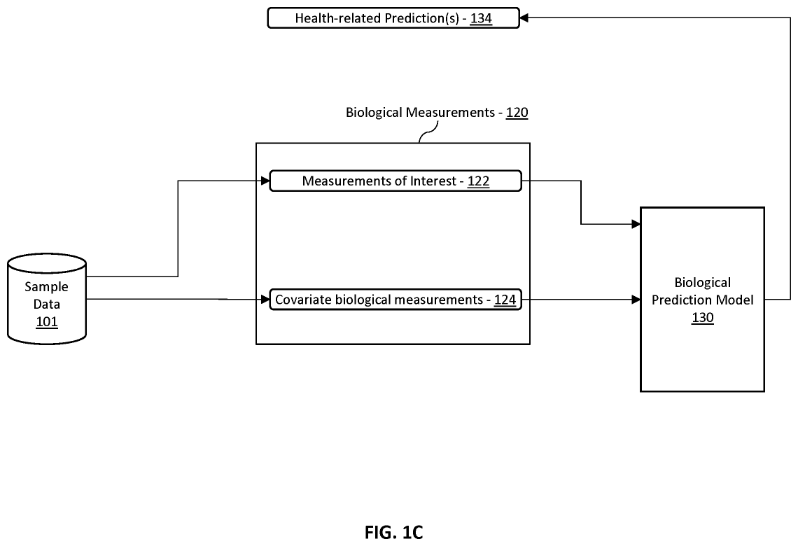

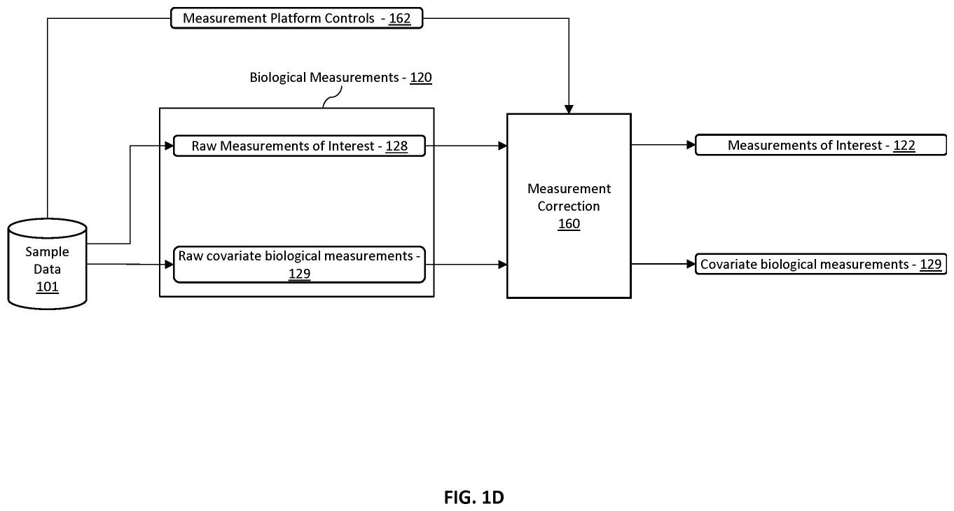

Referring now to FIG. 1A, FIG. 1B, FIG. 1C, and FIG. 1D, a block diagram is depicted that illustrates the use of a biological prediction model 130 in determining and correcting for noise in protein measurement based on one or more covariate measurements, in accordance with one or more embodiments of the present disclosure.

Proteomics platforms that measure protein amounts and other protein-based biomarkers can have measurement and/or pre-processing noise that can skew values in ways that can impede accuracy as well as comparison and/or measurement optimization with measurements from other systems. Similar issues in measurements can be found in other biologic omics fields, however, for illustrative purposes and ease of understanding, embodiments herein are detailed relative to proteomics. The principles detailed herein also apply to other omics.

Referring to FIG. 1A, an exemplary embodiment of a system is depicted that receives sample data 101 and generates inferred output(s) 132 using a biological prediction model 130. The sample data 101 may include biological measurements 110. The biological measurements 110 may include a first measurements group 112 and a second measurement group 114. In some embodiments, the system may process the sample data 101 to infer an expected measurement value for a measurement of interest from other biological measurements in the sample data 101.

In some embodiments, the sample data 101 may include measurements obtained from one or more biological samples. In some embodiments, the sample data 101 may include multi-omics data. The biological measurements 110 may represent traits, abundances, presence values, concentration values, effects, or other characteristics associated with a biological sample. Although FIG. 1A illustrates the biological measurements 110 as grouped into the first measurements group 112 and the second measurement group 114, the sample data 101 may include any suitable number of measurements groups, blocks, measurement types, or combinations thereof.

In some embodiments, the biological prediction model 130 may receive the first measurements group 112 and the second measurement group 114 as inputs. In some embodiments, the biological prediction model 130 may use predictive relationships among biological measurements to infer an expected measurement value for a selected biological measurement. The predictive relationships may include cross-biological measurement correlations between covariate biological measurements and a measurement of interest. The biological prediction model 130 may implement one or more machine learning techniques, such as a linear regression model, a non-linear regression model, a decision tree, boosting, a support-vector machine, a neural network, a neighbor-based algorithm, Naive Bayes, bagging, a random forest, or any combination thereof.

In some embodiments, the first measurements group 112 may include one or more biological measurements selected from the biological measurements 110. The second measurement group 114 may include one or more additional biological measurements selected from the biological measurements 110. In some embodiments, the first measurements group 112 and the second measurement group 114 may correspond to different blocks within the sample data 101. Two groups of measurements are depicted for understandability and illustration, but such an arrangement is non-limiting. Embodiments include two or more groups, such as 3, 4, 5, 6, 7, 8, 9, 10, or more groups of measurements. In some embodiments, the groups can include, e.g., different blocks, different measurement types, different omics modalities, or other semantically meaningful data groups within the sample data 101. For example, a group may include proteomic measurements obtained from a first block associated with a common reagent batch, assay plate, sample processing protocol, or other shared experimental condition, while another group may include proteomic measurements obtained from a different block. In some embodiments, a group may include measurements of a particular measurement type, such as protein abundance measurements, immunoassays, blood platelet counts, or nucleotide measurements. In some embodiments, a group may include measurements associated with a particular data modality or clinical category, such as genomics, proteomics, metabolomics, metagenomics, phenomics, transcriptomics, biomarkers, physiological measurements, anatomical measurements, clinical laboratory measurements, clinical covariates, demographics, diagnosis history, medication history, or other biological or phenotypic data. The biological prediction model 130 may process such groups separately and/or jointly to leverage cross-biological measurement correlations when inferring an expected measurement value.

In some embodiments, the different blocks may share a block control assay. The shared block control assay may introduce shared noise into the biological measurements 110. The biological prediction model 130 may process the first measurements group 112 and the second measurement group 114 to determine an expected measurement value notwithstanding measurement noise, internal error control noise, or other noise sources.

The inferred output(s) 132 may include one or more de-noised measurement values. In some embodiments, the inferred output(s) 132 may include a predicted de-noised measurement for a measurement of interest and, optionally, one or more de-noised covariate biological measurements. The system may use the inferred output(s) 132 to correct the sample data 101. For example, the system may overwrite a measurement value in the sample data 101 and/or modify a record associated with the measurement value to specify a de-noised measurement. In some embodiments, the system may use the inferred output(s) 132 for downstream prediction of health-related predictions, such as disease classification, disease risk, predicted molecular levels, predicted phenotypic traits, de-noised measurement data (e.g., of one or more of the groups of measurements), reconstructed or predicted measurement data (e.g., of one or more of the groups of measurements and/or for group(s) not input into the model, etc.), among other inferred outputs or any combination thereof.

Referring now to FIGS. 1B through 1D, in some embodiments, in order to address noise, such as measurement noise, internal error control noise, and other sources of noise, noise correction is provided to sample data 101, e.g., collected by a proteomics platform such as Olink™ or others. In some embodiments, the sample data 101 may be grouped in block data sets having biological measurements 120 from one or more samples. The block data sets relate to a set of proteins within a panel that are grouped according to a common or shared block control assay. However, the shared block control assay results in shared noise across proteins in the block data set. Thus, noise in one or more biological measurements of one or more measurement types (e.g., protein abundance, genetics, clinical lab data, among others or any combination thereof) can be corrected for noise with data from a same or different block data set. In some embodiments, techniques herein can be agnostic to blocks, correcting for noise regardless of whether the measurement(s) of interest 122 and/or the covariate biological measurement(s) 124 from a same, different or combination of block data sets.

In some embodiments, the block data sets may include data regarding each sample tested, including the measurement, a date of measurement, a reagent lot, a sample population demographic(s), among other attributes associated with each measurement. The block data sets may include one or more external reference measurements associated with each block, sample, plate, batch, or any combination thereof.

The block data sets may include in-band measurements, such as each measurement associated with sample, and/or out-of-band anchors, such as, e.g., serum CRP, hormone levels (e.g., testosterone, estradiol, etc.), and/or other chemistry panels, anthropometrics and/or demographics (e.g., age, sex, body-mass index (BMI), disease status, medication usage, etc.), omics data (e.g., NMR metabolite panels, methylation-based biomarkers, among other validated molecular data, etc.), among other data or any combination thereof.

Accordingly, in some embodiments, a measurement(s) of interest can be identified and used to determine, alone in combination with other measurement(s) of interest, a noise in the sample data 101. To do so, a measurement(s) of interest 122 can be identified and/or extracted. The measurement(s) of interest 122 may have a value that is correlated with one or more other biological measurements (“covariate” biological measurements 124). For example, the biological measurement may be influenced by or may influence a biological measurement of a correlated other biological measurement. Thus, the biological measurements of the correlated other measurement(s) may have predictive power for the measurement(s) of interest 122.

In some embodiments, the multi-omics platform(s) may include measurement correction 160 to reduce errors, noise, bias and other artifacts in the measurement data. For example, as shown in FIG. 1C, the sample data 101 may include raw measurement(s) of interest 128 and/or raw covariate biological measurement(s) 129 that may be corrected using measurement platform controls 162, such as block controls and other predefine measurement artifact controls or any combination thereof. Using the measurement platform controls 162, the measurement correction 160 may output the measurement(s) of interest 122 and/or the covariate biological measurement(s) 124. However, such measurement correction 160 may be insufficient, as detailed above, because noise may mimic some biological behaviors and mechanisms.

In some embodiments, the covariate biological measurements 124 can be identified and their predictive power on the measurement(s) of interest 122 can be leveraged, via a biological prediction model 130 to determine an expected measurement value for the measurement(s) of interest 122.

Therefore, in some embodiments, the covariate biological measurement(s) 124 can be used for their predictive power via correlation to the measurement(s) of interest 122. Thus, the covariate biological measurement(s) 124 can be input into a biological prediction model 130 trained to use the correlates to the measurement(s) of interest 122 in order to produce predicted de-noised measurement(s) 132 for the measurement(s) of interest 122, as well as other outputs, such as de-noised covariate biological measurement(s) 124, health-related prediction(s) 134 (e.g., classification of disease or disease risk, etc.), among other outputs or any combination thereof.

In some embodiments, the biological prediction model 130 of exemplary inventive computer-based systems and/or devices may be configured to utilize one or more exemplary AI/machine learning techniques chosen from, but not limited to, linear regression models, non-linear regression models, decision trees, boosting, support-vector machines, neural networks, nearest neighbor algorithms, Naive Bayes, bagging, random forests, and the like. In some embodiments and, optionally, in combination of any embodiment described above or below, an exemplary neural network technique may be one of, without limitation, feedforward neural network, radial basis function network, recurrent neural network, convolutional network (e.g., U-net) or other suitable network. In some embodiments and, optionally, in combination of any embodiment described above or below, an exemplary implementation of Neural Network may be executed as follows:
define Neural Network architecture/model,
transfer the input data to the exemplary neural network model,
train the exemplary model incrementally,
determine the accuracy for a specific number of timesteps,
apply the exemplary trained model to process the newly-received input data,
optionally and in parallel, continue to train the exemplary trained model with a predetermined periodicity.

In some embodiments and, optionally, in combination of any embodiment described above or below, the exemplary trained neural network model may specify a neural network by at least a neural network topology, a series of activation functions, and connection weights. For example, the topology of a neural network may include a configuration of nodes of the neural network and connections between such nodes. In some embodiments and, optionally, in combination of any embodiment described above or below, the exemplary trained neural network model may also be specified to include other parameters, including but not limited to, noise values/functions and/or aggregation functions. For example, an activation function of a node may be a step function, sine function, continuous or piecewise linear function, sigmoid function, hyperbolic tangent function, or other type of mathematical function that represents a threshold at which the node is activated. In some embodiments and, optionally, in combination of any embodiment described above or below, the exemplary aggregation function may be a mathematical function that combines (e.g., sum, product, etc.) input signals to the node. In some embodiments and, optionally, in combination of any embodiment described above or below, an output of the exemplary aggregation function may be used as input to the exemplary activation function. In some embodiments and, optionally, in combination of any embodiment described above or below, the noise may be a constant value or function that may be used by the aggregation function and/or the activation function to make the node more or less likely to be activated.

In some embodiments, because the biological prediction model 130 is trained to predict the de-noised measurement(s) 132 from covariate biological measurements 124 of proteins in block data sets different from the block data set of the measurement(s) of interest 122, the noise can be mitigated. Thus, the de-noised measurement(s) 132 may be the expected measurement(s) of interest 122 in the absence of noise. Accordingly, in some embodiments, the de-noised measurement(s) 132 can be used to correct the sample data 101, e.g., by overwriting the measurement(s) therein, and/or modifying a record of the measurement(s) to specify the de-noise measurement.

Similarly, in some embodiments, the biological prediction model 130 is trained to predict the de-noised measurement(s) 132 including de-noised covariate biological measurements corresponding to the covariate biological measurements 124. Thus, the biological prediction model 130 may mitigate noise in covariate data to the measurement(s) of interest 122. Thus, the de-noised measurement(s) 132 may be the expected covariate biological measurement(s) 124 in the absence of noise. Accordingly, in some embodiments, the de-noised measurement(s) 132 can be used to correct the sample data 101, e.g., by overwriting the measurement(s) therein, and/or modifying a record of the measurement(s) to specify the de-noise measurement.

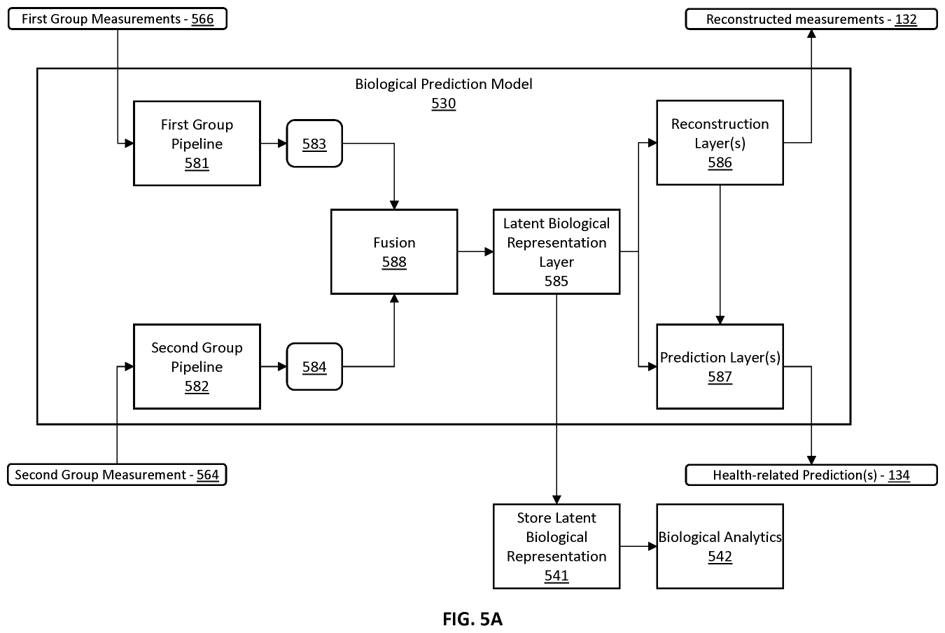

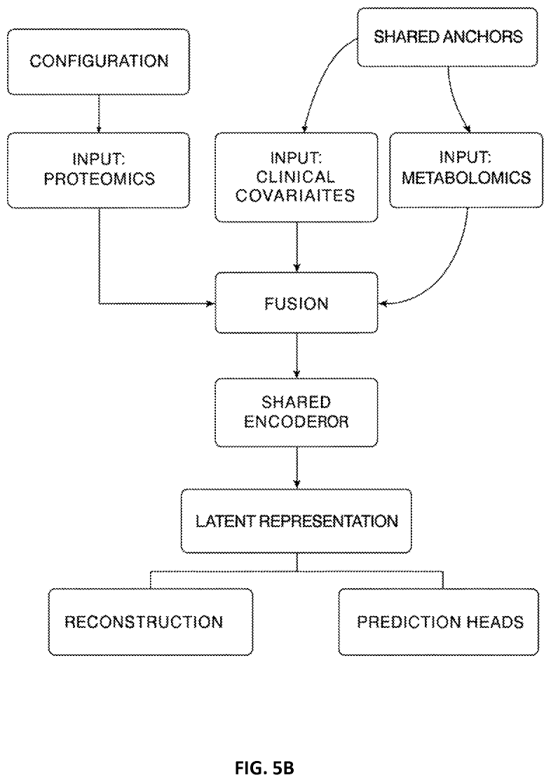

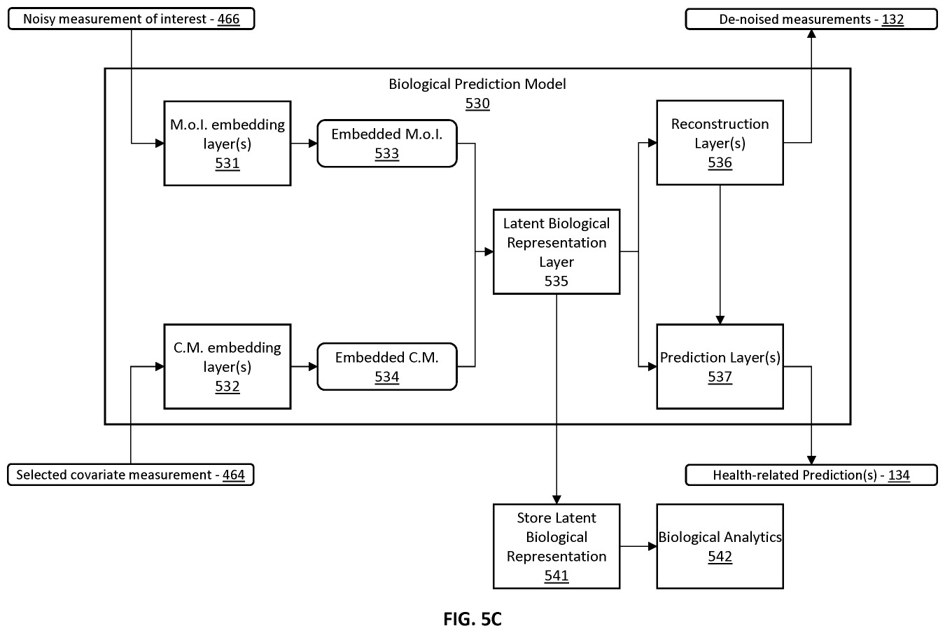

In some embodiments, as is described in greater detail below with reference to FIG. 5A-5C, the biological prediction model 130 may include layers configured and trained to use the de-noised measurement(s) 132 to predict a health-related prediction(s) 134. Such health-related prediction(s) 134 can include, e.g., a risk classification for one or more particular diseases, a classification of a most likely disease or disease state within a period of time, predicted molecular levels in the patient, predicted phenotypic traits within a patient, or other measurement and/or classification not otherwise provided in the measurement(s) of interest 122 and/or the covariate biological measurement(s) 124.

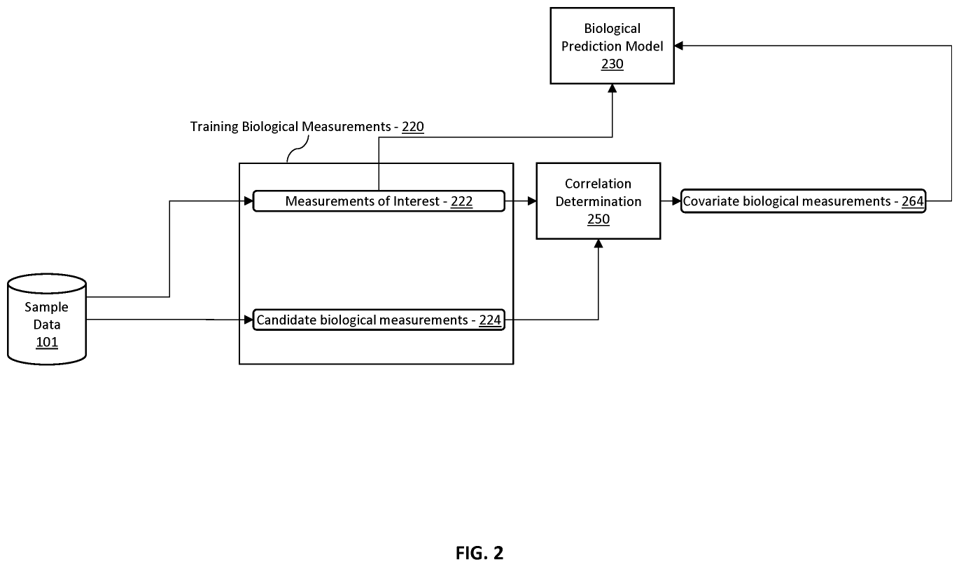

Referring now to FIG. 2, a block diagram is depicted that illustrates the identification of covariate biological measurements for use in training a biological prediction model 130 to output a predicted biological measurement for a particular measurement(s) of interest 122, in accordance with one or more embodiments of the present disclosure.

In some embodiments, a biological prediction model 230 can be trained on training sample data 230 having training biological measurements 220. Correlations between a measurement(s) of interest 222 and potential covariate biological measurements 224 can be determined and used to build a model that leverages the correlations to infer an expected biological measurement. Accordingly, techniques herein include obtaining, from a training data source, such as, e.g., the UKB or other validated set of biological data (e.g., biological measurements among other biological measurements), training data.

The sample data 101 relates to a set of biological measurements within a panel that are grouped according to a common or shared block control assay. However, the shared block control assay results in shared noise across biological measurements in the sample data 101. Thus, noise identified in one or more biological measurements of one or more biological measurements can be used to correct for noise.

Accordingly, in some embodiments, a model can be built for a measurement(s) of interest 122. To do so, a measurement(s) of interest 222 for the measurement(s) of interest 122 can be identified and/or extracted. The biological measurement 212 may have a value that is correlated with one or more other biological measurements. For example, the biological measurement 212 may be influenced by or may influence a biological measurement of a correlated other biological measurement. Thus, the biological measurements of the correlated other biological measurement(s) may have predictive power for the measurement(s) of interest 122.

Accordingly, in some embodiments, correlation determination 250 may be performed to identify a set of covariate biological measurements (e.g., correlated biological measurements) that influence the behavior of the measurement(s) of interest 122. To do so, the correlation determination 250 may obtain candidate biological measurements 224 from the biological measurements associated with the measurement(s) of interest 122 222.

The candidate biological measurements 224 for each candidate biological measurement can be analyzed for a potential correlation to the measurement(s) of interest 222 of interest. There is computational cost to the size of a model, and the more biological measurements used as covariate biological measurements can increase the size of the model. Thus, there is a balance between the added predictive power of the model due to an additional biological measurement used as a covariate biological measurement, and the computational costs of added the additional biological measurement. Accordingly, using all biological measurements to model the measurement(s) of interest 222 of interest may increase the size of the model without a commensurate benefit in performance or accuracy. Accordingly, the correlation determination 250 may determine a set of covariate biological measurements that maximize the performance of the model within a size constraint. For example, the correlation determination 250 may identify the top most correlation candidate biological measurements to the measurement(s) of interest 122. For example, the top most correlated candidate biological measurements may be, e.g., a top 10, 20, 30, 40, 50, 60, 70, 80, 90, 100, 150, 200, 250, 300, 350, 400, 450, 500 or more candidate biological measurements. In another example, the top most correlated candidate biological measurements may be, e.g., a top 75th percentile, 80th percentile, 85 percentile, 90th percentile, 95th percentile, 96th percentile, 97th percentile, 98th percentile, 99th percentile, or other percentile of candidate biological measurements.

In some embodiments, to select the covariate biological measurements, the correlation determination 250 may calculate the correlation coefficients between the measurement(s) of interest 122 and each of candidate covariate biological measurement.

The correlation coefficient is a measure that quantifies the strength of the linear relationship between two variables in a correlation analysis. For two variables, the formula compares the distance of each datapoint from the variable mean and uses this to explain how closely the relationship between the variables can be fit to a linear relationship describing the data. For example, the formula used can be, e.g., Pearson correlation, Intraclass Correlation, Spearman's rank, Kendall tau rank, Goodman and Kruskal's gamma, among others or any combination thereof.

For example, in some embodiments, the correlation determination 250 may iterate through each candidate biological measurement to assess the correlation between the corresponding corpus of candidate biological measurements 224 data of each candidate biological measurement to the biological measurements 222 of the measurement(s) of interest 122. After the correlation coefficient of each candidate biological measurement is determined, the correlation determination 250 may select the most correlated candidate biological measurements, and output the associated covariate biological measurements 264.

Using the covariate biological measurements 264, a model, such as a linear or non-linear model may be built that models the biological measurements 222 as a function of the covariate biological measurements 264. As a result, the biological prediction model 230 may be trained to enable prediction of an expected biological measurement for the measurement(s) of interest 122 based on the out-of-band covariate biological measurements. In some embodiments, a separate model may be built for each biological measurement in the training sample data 201.

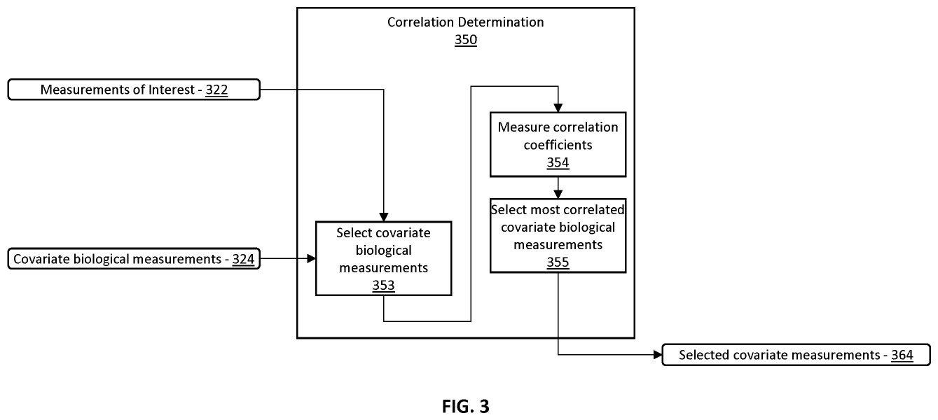

Referring now to FIG. 3, another block diagram is depicted that illustrates the identification of covariate biological measurements that are correlated to biological measurement values of a particular measurement(s) of interest 322, in accordance with one or more embodiments of the present disclosure.

The Biological measurements 322 represent the actual measured values of a measurement(s) of interest 322 within a specific block, which are subject to noise introduced by shared block controls. These measurements serve as the baseline data from which correlations with covariate biological measurements are assessed. The biological measurements are important for identifying systematic noise and for training models that predict expected biological measurement values when such noise are not present.

The Covariate Biological measurements 324 may include measurements taken from biological measurements that of a different type (e.g., “out-of-band”) from the measurement(s) of interest 322, thus providing an external reference that is not subject to the same noise characteristics. By analyzing these covariate biological measurements, the system can identify biological measurements that have a predictive relationship with the measurement(s) of interest 322. These relationships are leveraged to adjust the biological measurements, thereby reducing the impact of noise. The selection of appropriate covariate biological measurements is important, as this choice influences the accuracy of the predicted biological measurements.

The Correlation Determination 350 component is a mechanism that identifies and quantifies the relationships between the measurement(s) of interest 322 and potential covariate biological measurements. This component involves several sub-processes, including the selection of covariate biological measurements 353. The correlation determination process measures correlation coefficients 354 to assess the strength of the relationships between the measurement(s) of interest 322 and candidate covariate biological measurements. The most correlated covariate biological measurements are then selected 355 for use in the prediction model. This process can ensure that the prediction model is based on the most relevant and informative data.

The selected covariate biological measurements 364 represent the biological measurements identified as having a significant correlation with the measurement(s) of interest 322. These biological measurements are selected based on their correlation coefficients, which indicate the strength and direction of their relationship with the measurement(s) of interest 322. The selected covariate biological measurements are used as inputs to the biological prediction model, providing the data needed to predict the expected biological measurement values. The selection of these biological measurements can influence the effectiveness of the noise correction and the accuracy of the predicted biological measurements.

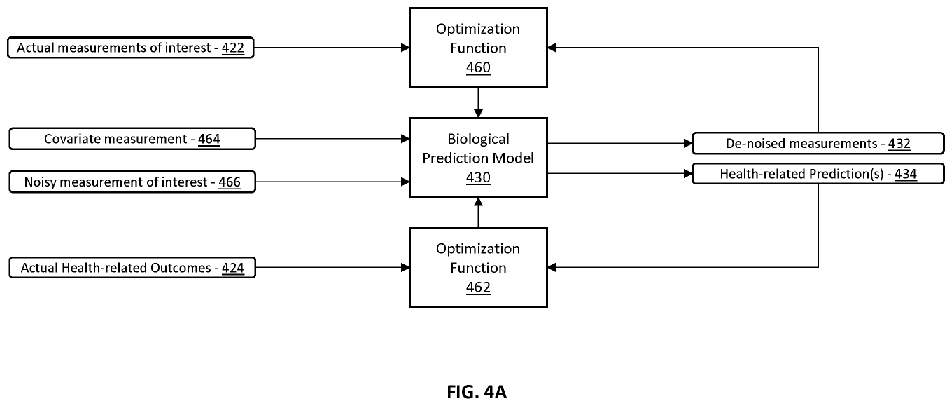

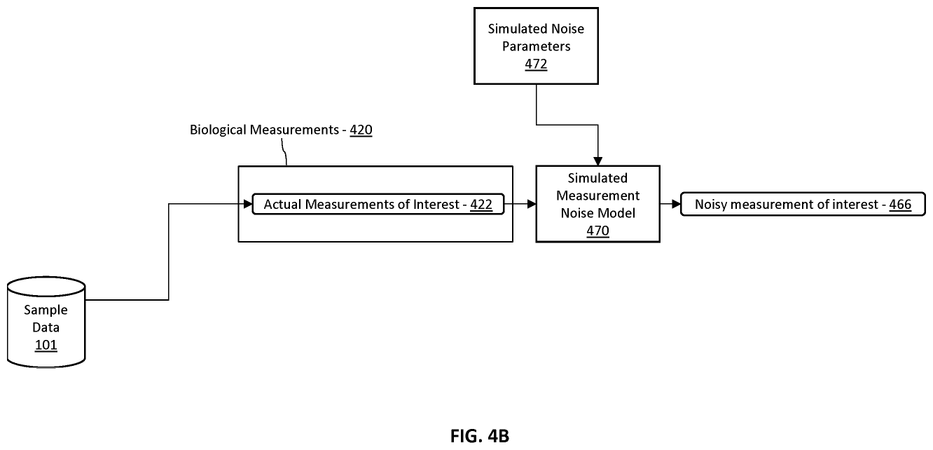

Referring to FIG. 4A, a block diagram is depicted that illustrates the interaction between biological measurements, covariate biological measurements, and a prediction model with one or more optimization functions in accordance with one or more embodiments of the present disclosure.

In some embodiments, the biological prediction model 430 to predict a de-noised measurement(s) 432 and a health-related prediction(s) 434 for a particular noisy measurement(s) of interest 466 and covariate measurements 464. In some embodiments, the biological prediction model 430 ingests a feature vector that encodes features representative of biological measurement values of noisy measurement interest 464 and covariate biological measurements 464, e.g., as detailed above with reference to FIG. 3. In some embodiments, the biological prediction model 430 processes the feature vector with parameters to produce a prediction of the de-noised biological measurement(s) 432 and the health-related prediction(s) 434. In some embodiments, the health-related prediction(s) 434 can include diagnostic predictions (e.g., risk of a disease, prognosis, disease classification, etc.), phenotypic measure, biomarker(s), among other measurements, metrics, analyses and other health-related predictions or any combination thereof.

In some embodiments, the parameters of the biological prediction model 430 may be implemented in a suitable machine learning model including a prediction machine learning model, such as, e.g., Linear Regression, Logistic Regression, Ridge Regression, Lasso Regression, Polynomial Regression, Bayesian Linear Regression (e.g., Naive Bayes regression), a convolutional neural network (CNN), a recurrent neural network (RNN), decision trees, random forest, support vector machine (SVM), K-Nearest Neighbors, or any other suitable algorithm for predicting output values based on input values.

In some embodiments, the biological prediction model 430 processes the features encoded in the feature vector by applying the parameters of the prediction machine learning model to produce a model output vector. In some embodiments, the model output vector may be decoded to generate one or more numerical output values indicative of each of the de-noised biological measurement(s) 432 and the health-related prediction(s) 434. In some embodiments, the model output vector may include or may be decoded to reveal the output value(s) based on a modelled correlation between the feature vector and a target output. In some embodiments, the numerical output may represent the biological measurement value(s) for the de-noised biological measurement 432 for the particular noisy measurement(s) of interest 422 and/or for the covariate measurement(s) 464.

In some embodiments, the parameters of the biological prediction model 430 may be trained based on known outputs. For example, the biological measurement values for the noisy measurement(s) of interest 466 and covariate measurements 464 may be paired with a target value or known value to form a training pair, such as an actual measurement of interest 422 and/or actual health-related outcome(s) 424 based on an observed result and/or human annotated value representing a data point in the relationship between the historical biological measurement values and the de-noised biological measurement(s) 432. In some embodiments, the biological measurement values for the noisy measurement(s) of interest 466 and covariate measurements 464 may be provided to the biological prediction model 430, e.g., encoded in a feature vector, to produce a predicted output value. In some embodiments, an optimization function 460 associated with the biological prediction model 430 may then compare the predicted de-noised measurement(s) 432 with the known output of a training pair including the actual measurement(s) of interest 422 to determine an error of the predicted output value. Similarly, an optimization function 462 associated with the biological prediction model 430 may also compare the health-related prediction(s) 434 with the known output of a training pair including the actual health-related outcome(s) 424 to determine an error of the predicted output value In some embodiments, the optimization function 460 may employ a loss function, such as, e.g., Hinge Loss, Multi-class SVM Loss, Cross Entropy Loss, Negative Log Likelihood, or other suitable classification loss function to determine the error of the predicted output value based on the known output.

In some embodiments, the known output may be obtained after the biological prediction model 430 produces the prediction, such as in online learning scenarios. In such a scenario, the biological prediction model 430 may receive the biological measurement values for covariate biological measurements 464 and generate the model output vector to produce an output value representing the biological measurement value(s) for the de-noised biological measurement(s) 432. Subsequently, a user may provide feedback by, e.g., modifying, adjusting, removing, and/or verifying the output value via a suitable feedback mechanism, such as a user interface device (e.g., keyboard, mouse, touch screen, user interface, or other interface mechanism of a user device or any suitable combination thereof). The feedback may be paired with the biological measurement values for the covariate biological measurement(s) 464 to form the training pair and the optimization function 460 may determine an error of the predicted output value using the feedback.

In some embodiments, based on the error, the optimization functions 460 and/or 462 may update the parameters of the biological prediction model 430 using a suitable training algorithm such as, e.g., backpropagation for a prediction machine learning model. In some embodiments, backpropagation may include any suitable minimization algorithm such as a gradient method of the loss function with respect to the weights of the prediction machine learning model. Examples of suitable gradient methods include, e.g., stochastic gradient descent, batch gradient descent, mini-batch gradient descent, or other suitable gradient descent technique. As a result, the optimization function 460 may update the parameters of the biological prediction model 430 based on the error of predicted labels in order to train the biological prediction model 430 to model the correlation between: biological measurement values for the noisy measurement(s) of interest 466 and covariate biological measurement(s) 464, and de-noised biological measurement 432 and/or health-related prediction(s) 434 in order to produce more accurate output values based on biological measurement values for covariate biological measurements 464.

Referring to FIG. 4B, a block diagram is depicted that illustrates the creation of training data for training the biological prediction model, using simulated noise of biological measurements, to reconstruct the measurement(s) without the noise in accordance with one or more embodiments of the present disclosure.

In some embodiments, training data for the biological prediction model 430 can be scarce or otherwise difficult to obtain (e.g., costly to perform the measurements and manually verify both noisy and de-noised versions and/or annotate the data, processor and memory intensive to scrape and/or format previously acquired data having both noisy and de-noised data, etc.). Thus, to efficiently train the biological prediction model 430, noise may be simulated and imposed on measured data, thus providing a version of the data with noise and without noise to use as the raw and actual data, respectively in a training pair. In some embodiments, the simulated noise may be structured to simulate the correlation structures of technical and systematic noise in the omics data, e.g., based on known, measured or simulated noise introduced by the multi-omics platform.

Accordingly, the biological measurements 420 of the sample data 101, including the measurement(s) of interest 422, can be provided to a simulated measurement model 470. In some embodiments, the simulated measurement model 470 can include one or more algorithm, statistical and/or machine learning models configured to simulate the noise based on the type and values of the measurements of each of the measurement(s) of interest 422.

In some embodiments, the simulated measurement model 470 can access or other otherwise include configurations for noise simulation, including simulated noise parameters 472 mapping transformations of the measurement(s) based on the measurement type and/or the measurement values. For example, the simulated measurement model 470 can include a random value generator or pseudo random value generator that uses the measurement values and a probability distribution as inputs in order to output a random or pseudo-random modification to the measurement values. The simulated noise parameters 472 can map the probability distribution to the type of measurement (e.g., protein abundance, cell count, microbe abundance of a microbiome, among other types of multi-omics measurements or any combination thereof) including, e.g., a mean and/or variance associated with the probability distribution specific to that type of measurement.

In some embodiments, the simulate the noise, the total simulated noise variance (relative to the total variance of the training data) that is to be added to the training data can be specified as a scalar value between 0.0 and 1.0, where 1.0 represents noise variance equal to 100% of the total variance of the training dataset.

The noise variance is partitioned across various “channels” according to a specified set of sharing weights. A channel can correspond to one of: batches, plates, samples, blocks of measurements, individual measurements, unstructured or other groupings or any combination thereof.

Samples are assigned randomly to a fixed set of plates; plates to a fixed set of batches; measurements to a fixed set of blocks. Unstructured applies to any measurement of any particular measurable item (e.g., any protein) in any sample.

For example, we might allocate 20% of the total noise budget may be allocated to the plates channel. The total noise budget may be, e.g., 5%, 10%, 15%, 20%, 25%, 30% or other percentage in a range of about 5 to 50 percent. As a result, all the measurement values belonging to a particular plate (regardless of which sample, measurement, or other channel) receives a distinct random level of noise, the combined variance of which would sum to the total noise budget, thus creating a synthetic technical noise component correlated with plate membership.

In some embodiments, a set of noisy replicates from original (presumed noisy) samples can be generated to create a training dataset of noisy samples, including creating a quantity (N) (a configurable number) of copies of each original sample (e.g., the vector of all of the sample's measurements, including proteins, clinical values, etc.). Using the structured noise variance parameters (i.e., total variance and channel shares of total), generate and apply noise to all the measurements (such as the omics measurements, e.g., the measurement(s) of interest 422 and, optionally, other covariate measurement(s) 464).

As a result, the simulated measurement model 470 can ingest the values of the measurement(s) of interest 422, simulate noise associated with type(s) of the measurement(s) of interest 422, and augment the measurement(s) of interest 422 to apply the noise in order to output noisy measurement(s) of interest 466 and/or noisy covariate biological measurement(s) 466. Accordingly, the simulated noisy measurements can be paired with the original measurements prior to the injection of noise for use as a training pair in training the biological prediction model 430, e.g., as detailed with respect to FIG. 4A above.

Referring to FIG. 5A, a biological prediction model 530 may implement a machine-learning architecture configured to integrate heterogeneous biomedical data sources into a unified predictive representation. The machine-learning architecture may operate in environments in which patient information is organized into distinct feature groups, such as demographics, laboratory measurements, proteomic panels, metabolomic panels, imaging-derived values, diagnosis histories, medication histories, and other clinically meaningful data planes. In some embodiments, the biological prediction model 530 may preserve semantic identity of each feature group while learning a shared latent representation that supports reconstruction, prediction, interpretability, and graceful degradation when one or more feature groups are unavailable.

In some embodiments, the machine-learning architecture may include a learned set of shared concept anchors. The shared concept anchors may define a common slot-based semantic basis that is reused across multiple input groups. The biological prediction model 530 may interpret each input group relative to the shared concept anchors rather than collapsing multiple modalities into a single undifferentiated vector at an initial stage. Such an arrangement may create a common representational coordinate system in which different input groups may express shared information and group-specific information. In some embodiments, the shared concept anchors may be regularized to remain diverse and non-redundant so that the slot basis maintains distinct semantic directions. The shared concept anchors may therefore provide a semantic scaffold that allows multiple heterogeneous data groups to project into a shared space while maintaining aligned conceptual axes across groups.

In some embodiments, each input group may be processed through a corresponding group pipeline, including a respective group adapter, pooling (e.g., attention-based pooling), group gating, group dropout, among other stages or any combination thereof, to produce a respective encoded representation 583 and 584, respectively. For example, the first group pipeline 581 may process first group measurements 566 to produce a first group encoding 583, and the second group pipeline 582 may process second group measurement 564 to produce a second group encoding 584. A corresponding group adapter may convert raw features and/or normalized features from a respective input group into an internal token representation and may refine the internal token representation using intra-group contextual modeling. In some embodiments, intra-group contextual modeling may be performed using a compact transformer-style mechanism, although other context-sensitive encoders may be used. Such processing may allow the biological prediction model 530 to model each input group on group-specific terms and to learn within-group relationships before cross-group fusion occurs. Such an arrangement may be advantageous for input groups having constituent features that are correlated, partially redundant, hierarchically organized, and/or differently informative in different contexts.

After per-group contextualization, each group encoding 583 and 584 may interact with the shared concept anchors through an attention-based pooling mechanism. In some embodiments, the shared concept anchors may act as learned queries, and group features may act as keys and values. Such processing may generate, for each input group, a set of slot-wise deviation vectors that describe modification of the shared semantic basis by the corresponding input group. A resulting group representation may therefore include the shared concept anchors and a learned group-specific deviation. Accordingly, the biological prediction model 530 may translate each input group into a common slot language that remains directly comparable across groups while preserving semantic identity of each input group.

In some embodiments, the machine-learning architecture may further include a group gating mechanism. A learned gate may be associated with each input group and may scale a magnitude of contribution from the corresponding group representation to a shared representation. The learned gate may represent an adaptive confidence factor, salience factor, and/or influence factor for the corresponding input group. Such gating may allow the biological prediction model 530 to represent both informational content of an input group and relative influence of the input group on a fused latent state. In some embodiments, learned gates may be regularized to discourage degenerate solutions in which a single input group dominates the biological prediction model 530 while one or more additional input groups contribute limited information. Such gating may improve training stability and may also support downstream interpretability by providing an explicit measure of contribution from each data plane.

In some embodiments, the biological prediction model 530 may perform group dropout during training to support robustness under missing modalities, unavailable records, or intentional feature ablation. During group dropout, one or more input groups may be stochastically replaced with an anchor-only representation. Because multiple input groups may map into a common slot basis, fusion 588 may generate a structurally valid fused representation even when one or more input groups are removed, missing, or withheld. Such training may encourage the biological prediction model 530 to distribute information across multiple input groups in a redundant and organized manner and may improve resilience in settings in which one or more assays, measurements, or records are unavailable.

In some embodiments, fusion 588 may operate at a level of group contributions within the shared slot space. The biological prediction model 530 may fuse group-specific deviations without repeatedly reintroducing the shared concept anchors from each input group. For example, the shared concept anchors may be added once, while group-specific deviations may be aggregated through a learned fusion mechanism and/or a structured fusion mechanism. Such an arrangement may reduce double-counting of common structure and may cause each input group to contribute incremental information relative to the shared semantic basis. In some embodiments, a fused slot representation may be passed through a shared encoder configured to refine interactions among slots and to generate a globally integrated representation spanning available input groups.

In some embodiments, a latent biological representation layer 585 to create a latent biological representation, which may convert encoded slots into one or more compact latent vectors suitable for downstream tasks. The latent biological representation layer 585 may use averaging, designated summary-slot pooling, learned attention pooling, and/or any other suitable slot-pooling strategy. The resulting latent representation may serve as a substrate for multiple downstream heads. For example, reconstruction layer(s) 586 may reconstruct one or more source groups, and prediction layer(s) 587 may generate health-related prediction(s) 134, such as clinical outcome predictions, disease-state classifications, continuous phenotype estimates, survival-related predictions, treatment-response predictions, and other supervised outputs. In some embodiments, the machine-learning architecture may support multiple output heads simultaneously so that a single integrated representation supports reconstruction and prediction in parallel.

In some embodiments, reconstruction layer(s) 586 may reconstruct one or more observed groups from shared representations and/or partially shared representations. Reconstruction may include denoising reconstruction in which a subset of observed entries is intentionally corrupted or masked during training and reconstruction layer(s) 586 recover corresponding values. Such training may encourage a latent state to capture structured cross-group dependencies rather than memorizing input values. Reconstruction may occur within a group, across groups, and/or from intermediate representations, thereby promoting a latent space that is generative, robust, and semantically aligned.

In some embodiments, the machine-learning architecture may support deep supervision at multiple representational levels. Auxiliary prediction heads and/or reconstruction heads may be attached to a final latent state, a group-level representation, a fusion-level representation, an encoder-level representation, and/or another intermediate representation. Such auxiliary supervision may provide additional gradient pathways during training and may encourage intermediate representations to remain semantically useful. Such an arrangement may be particularly beneficial in architectures configured to preserve modularity and robustness under group ablation.

In some embodiments, regularization may shape internal organization of the machine-learning architecture. For example, the shared concept anchors may be regularized for diversity, shared slots may be regularized for independence and/or decorrelation, latent coordinates may be regularized to reduce entanglement, and learned gates may be regularized for balanced utilization. In some embodiments, cross-group slot consistency penalties may encourage semantically aligned behavior among same-index slots from different input groups. Such regularization may bias the biological prediction model 530 toward a factorized and interpretable internal structure in which individual slots and latent dimensions correspond to relatively distinct concepts.

In some embodiments, the machine-learning architecture may further support internal attribution. Because the biological prediction model 530 may explicitly model contributions from features within an input group to shared slots and contributions from shared slots to outputs, the biological prediction model 530 may provide attribution paths from features to slots to outputs. Such an arrangement may support intrinsic interpretability without requiring a separate post hoc explainer. For example, the biological prediction model 530 may identify features within a particular input group that contribute to a particular prediction and may further identify semantic slots through which corresponding contributions propagate.

In some embodiments, the machine-learning architecture may also support ablation-based analysis. Because each input group may contribute through a modular slot-aligned pathway, the biological prediction model 530 may measure an effect of removing or attenuating an input group without retraining an entire model. Such analysis may support evaluation of marginal utility for individual data planes and combinations of data planes and may assist scientific analysis, cost-benefit assessment, and deployment planning in environments having expensive or inconsistently available modalities.

In some embodiments, training of the biological prediction model 530 may include multi-objective optimization over reconstruction losses, denoising losses, predictive losses, and architectural regularizers. Training may further include annealing schedules, including gradual reduction of group dropout over time, adaptive learning-rate strategies, early stopping procedures, and loss balancing across multiple outputs. Such training may shape a representation that is robust, modular, interpretable, and transferable across varying subsets of available data. In some embodiments, model selection may consider predictive quality and stability under differing input conditions, including partial-group conditions.

Accordingly, the machine-learning architecture may combine a common learned anchor basis for heterogeneous biomedical groups, group-specific deviations relative to the common learned anchor basis, explicit gating and dropout-based robustness training for controlling group influence, integration of shared slot representations into a compact latent state supporting multiple supervised and reconstructive tasks, and structured interpretability through maintained paths from raw features to shared semantic slots to outputs. Although the foregoing description emphasizes biomedical data, the same architecture may be applied to other multimodal and multi-group learning problems in which aligned latent semantics, robustness to missing groups, and interpretable group contributions may be desirable.

FIG. 5B shows a block diagram illustrating a biological prediction model architecture in which grouped measurements are processed to generate a latent biological representation for downstream prediction. In the illustrated embodiment, the biological prediction model architecture may include a configuration 501, shared anchors 502, input proteomics 503, input clinical covariates 504, input metabolomics 505, fusion 506, a shared encoder 507, a latent representation 508, and prediction heads 509. As shown, the architecture may receive multiple semantically meaningful input groups, including proteomics, clinical covariates, and metabolomics, and may preserve semantic identity of the respective input groups while mapping the respective input groups into a shared internal basis.

The configuration 501 may specify one or more operating parameters for the biological prediction model architecture. In some embodiments, the configuration 501 may define aspects of grouped processing, shared-anchor processing, fusion behavior, encoder operation, latent projection, output selection, regularization, training settings, or any combination thereof. As illustrated, the configuration 501 is associated with the input proteomics 503. Such an arrangement may indicate that processing of the input proteomics 503 is controlled, initialized, constrained, and/or otherwise parameterized according to the configuration 501.

The shared anchors 502 may define a common internal representational basis reused across multiple input groups. In some embodiments, the shared anchors 502 may be implemented as shared concept anchors and may provide a common slot-based semantic basis for the grouped measurements. As shown, the shared anchors 502 are coupled with the input clinical covariates 504 and the input metabolomics 505. In some embodiments, the shared anchors 502 may additionally be used with the input proteomics 503. The shared anchors 502 may permit different input groups to express shared information and group-specific information relative to aligned conceptual axes rather than being collapsed into a single undifferentiated vector at an initial stage.

The input proteomics 503, the input clinical covariates 504, and the input metabolomics 505 may correspond to different grouped measurements provided to the biological prediction model architecture. In some embodiments, the grouped measurements may include biological and clinical data organized into distinct feature groups. The input proteomics 503 may include proteomic measurements, the input clinical covariates 504 may include clinical laboratory measurements, demographics, diagnosis information, medication information, and/or other clinical covariates, and the input metabolomics 505 may include metabolomic measurements. In some embodiments, each input group may be processed through a corresponding group pipeline so that within-group relationships may be learned before cross-group fusion occurs.

The fusion 506 may receive group contributions from the input proteomics 503, the input clinical covariates 504, and the input metabolomics 505. In some embodiments, each input group may interact with the shared anchors 502 through pooling, including attention-based pooling, to generate a group representation in a shared slot space. The fusion 506 may aggregate group-specific deviations relative to the shared anchors 502 without repeatedly reintroducing the shared anchors 502 from each input group. Such an arrangement may reduce double-counting of common structure and may permit each input group to contribute incremental information relative to the shared semantic basis. In some embodiments, the fusion 506 may be configured to remain structurally valid when one or more input groups are removed, missing, or withheld.

The shared encoder 507 may receive a fused representation from the fusion 506 and may refine interactions among slots or other encoded elements spanning the available input groups. In some embodiments, the shared encoder 507 may generate a globally integrated representation based on the grouped measurements. The latent representation 508 may be generated from the output of the shared encoder 507 and may include one or more compact latent vectors suitable for downstream tasks. In some embodiments, the latent representation 508 may serve as a substrate for reconstruction and prediction in parallel and may be stored or used for downstream biological analytics, interpretation, and/or further modeling.

The prediction heads 509 may receive the latent representation 508 and may generate one or more outputs. In some embodiments, the prediction heads 509 may include multiple output heads operating simultaneously on the latent representation 508. The outputs may include health-related predictions, including clinical outcome predictions, disease-state classifications, continuous phenotype estimates, survival-related predictions, treatment-response predictions, and/or other supervised outputs. In some embodiments, the biological prediction model architecture illustrated in FIG. 5B may also support reconstruction of one or more source groups from shared representations and/or partially shared representations, thereby enabling prediction and reconstruction from a common latent biological representation.

Referring to FIG. 5C, a block diagram illustrates an exemplary implementation of the biological prediction model utilizing deep neural network machine learning to output de-noised biological measurements and/or health-related predictions based on grouped multimodal measurement data including a measurement of interest and covariate measurements in accordance with one or more embodiments of the present disclosure.

In some embodiments, an example of the biological prediction model 130 (such as the biological prediction model 230 and/or biological prediction model 430) can include a deep neural network (DNN)-based biological prediction model 530. The DNN-based biological prediction model 530 can include layers architected to allow for the predictive generation of de-noised measurements 132 as well as health-related prediction(s) 134 in a one-shot or single stage approach (e.g., via one run of an instance of the biological prediction model 530). Thus, rather than multiple models being used to separately generate the de-noised measurements 132 and health-related prediction(s) 134 (e.g., an independent model for each measurement being de-noised and each health-related prediction), the biological measurement model 530 can utilize a particular arrangement of the neural network layers to produce all of the outputs in one run for a particular measurement of interest and its associated covariate measurements. To do so, for example, the DNN-based biological prediction model 530 can include input layers including measurement-of-interest (M.o.I.) embeddings layer(s) 531 and covariate measurement (C.M.) embedding layer(s) 532, latent biological representation layer(s) 535, and output layers include reconstruction layer(s) 536 and prediction layer(s) 537.

In some embodiments, the model structure of the biological prediction model 530 can be a multi-feature channel, multi-output channel feed-forward deep neural network having multiple internal paths including varying/configurable numbers of hidden layers of varying/configurable sizes. The feed-forward paths of the biological prediction model 530 can include:
omics (protein) feature inputs processed through hidden layers (e.g., the M.o.I. embeddings layer(s) 531) and then through latent layer(s);
covariates (contemporaneous clinical) feature inputs processed through hidden layers (e.g., the C.M. embedding layer(s) 532) and then through latent layer(s);
concatenated latent layers processed through hidden layers and then through reconstructed omics (proteins);
concatenated latent layers processed through hidden layers and then through reconstructed covariates;
concatenated latent layers processed through hidden layers and then through to predicted phenotypic/other clinical outputs and/or could also concatenate latent layers with reconstructed omics;
concatenated latent layers processed through hidden layers and then through to predicted disease risk and/or could also concatenate latent layers with reconstructed omics;

In some embodiments, the layers themselves of the DNN structure may include arrays of identical linear nodes, typically having activation functions (e.g., sigmoid activation functions) except for regression outputs (e.g., where the output can be a real number).

In some embodiments, separate embedding layers for each of the M.o.I. and the C.M. are shown, resulting in separate M.o.I. embeddings 533 and C.M. embeddings 534. Such an arrangement is illustrative and non-limiting. Indeed, the noisy measurement(s) of interest 466 and the selected covariate measurement(s) 464 can be combined, e.g., concatenated or other form of combination, into a single input to the biological prediction model 530 for embedding by a single embedding layer.

In some embodiments, the M.o.I. embedding layer(s) 531 can ingest the noisy measurement interest 466 and produce embedded M.o.I. 533. In some embodiments, the M.o.I. embedding layer(s) 531 can include one or more layers or neurons, each neuron including a respective activation function and a respective weight such that the weight of each neuron in each layer can be trained to encode the values of the noisy measurement(s) of interest 466 into a feature space that is of lower dimensions than the noisy measurement(s) of interest 466. For example, noisy measurement(s) of interest 466 may exist multi-dimensional data including a time dimension, an amplitude value, and other measurements for each measurement of interest, which can result in multi-dimensional data, particularly where multiple measurements of interest are being ingested at a time (e.g., multiple protein abundances being de-noised in a run of the model). Thus, the M.o.I. embedding layer(s) 531 may encode the noisy measurement(s) of interest 466 into a lower (e.g., one or two dimension) representation.

The M.o.I. embedding layer(s) 531 may use one or more layers of the neurons to create the embedded M.o.I. 533. The layers may be fully connected or partially connected, or may be a single layer. Moreover, the layers can be arranged as convolutional layers, recurrent layers, attention layers and/or transformer layers or a combination thereof. Together, the layers may be constructed to form a particular embedding algorithm or model, such as, e.g., locally linear embedding (LLE), DeepWalk, node2vec, among others or any combination thereof.

Similarly, in some embodiments, the C.M. embedding layer(s) 532 can ingest the noisy covariate measurement 464 and produce embedding C.M. 534. In some embodiments, the C.M. embedding layer(s) 532 can include one or more layers or neurons, each neuron including a respective activation function and a respective weight such that the weight of each neuron in each layer can be trained to encode the values of the noisy covariate measurement(s) 464 into a feature space that is of lower dimensions than the noisy covariate measurement(s) 464. For example, noisy covariate measurement(s) 464 may exist multi-dimensional data including a time dimension, an amplitude value, and other measurements for each biological measurement, which can result in multi-dimensional data, particularly where multiple biological measurements are being ingested at a time (e.g., multiple covariate measurements to the protein abundances being de-noised in a run of the model). Thus, the C.M. embedding layer(s) 532 may encode the noisy covariate measurement(s) 464 into a lower (e.g., one or two dimension) representation.

The C.M. embedding layer(s) 532 may use one or more layers of the neurons to create the embedding C.M. 534. The layers may be fully connected or partially connected, or may be a single layer. Moreover, the layers can be arranged as convolutional layers, recurrent layers, attention layers and/or transformer layers or a combination thereof. Together, the layers may be constructed to form a particular embedding algorithm or model, such as, e.g., locally linear embedding (LLE), DeepWalk, node2vec, among others or any combination thereof.

In some embodiments, the latent biological representation layer 535 may include one or more hidden layers of the deep neural network that ingest the embedded M.o.I. 533 and the embedded C.M. 534 and produce a latent biological representation representative of the sample from which the input measurements are collected. To do so, in some examples, the latent biological representation layer 535 can include one or more convolutional and/or recurrent layers of nodes that compress the embedded M.o.I. 533 and the embedded C.M. 534 into a latent space. The latent space can encode features of the embedded M.o.I. 533 and the embedded C.M. 534, including relationships between the embedded M.o.I. 533 and the embedded C.M. 534. To do so, the nodes may include one or more node types, such as long-short term memory (LSTM), perceptrons, gated recurrent units (GRU) among others or any combination thereof. Moreover, the layers can be arranged as convolutional layers, recurrent layers, attention layers and/or transformer layers or a combination thereof.

In some embodiments, the biological prediction model 530 may output the latent biological representation to memory to store the latent biological representation 541 for other uses. For example, the latent biological representation, as a representation of features of sample based on the noisy measurement of interest 466 and the covariate measurement 464, can be used in downstream biological analytics 542, including statistical analytics, other machine learning models (e.g., for predictive model of particular diseases and/or parameters, classification of samples, among other modelling or any combination thereof), as well as other uses.

In some embodiments, the latent biological representation 541 may embody an encoded representation of biological features, the latent biological representation 541 on its own can be extracted for purposes of interpretation or further modeling. For example, the latent biological representation 541 may be used to ascertain in which ways the outputs of the latent layer combine the input feature space (via any hidden layers) to discover composite features that capture different aspects of biology; and discover how these correspond to known systems, pathways, etc. In another example, the latent biological representation 541 can, once trained, be fixed and used as the foundation for a new model whose purpose is to predict a different set of target outputs or to specialize further in a particular domain, such as, without limitation, a latent layer trained to predict a broad set of disease risks (and thus learn a representation of general biology) could be augmented with trainable downstream layers to construct and train a new model specialized in a particular disease area (e.g., neurological). In another example, the latent biological representation 541 can be used to observe how the latent features change as the feature input values change in order, e.g., discover patterns; infer what sorts of feature inputs might result in desired latent feature outputs, among other analyses or any combination thereof.

In some embodiments, the output layers of the biological prediction model 530 can include sets of layers for each type of output to be produced. For example, the reconstruction layer(s) 536 can include layers for reconstructing de-noised measurement(s) of interest and/or reconstructing de-noised covariate measurement(s) 464. Similarly, the output layers can include sets of layers as prediction(s) layers 537 for each type of prediction being made, such as, e.g., diagnostic classification of one or more diseases, disease risk score(s), predictions of unmeasured data such as molecular levels, phenotypic traits, genetic traits, biometrics, among others or any combination thereof. In some embodiments the prediction layer(s) 537 may use both the latent biological representation produced by the latent biological representation layer(s) 535 as well as the de-noised measurement(s) 132 produced by the reconstruction layer(s) 536.

In some embodiments, to produce each output, each set of output layers may be include one or more layers or neurons, each neuron including a respective activation function and a respective weight such that the weight of each neuron in each layer can be trained to decode the latent representation of the noisy measurements from the latent space. For example, as detailed above, the latent biological representation may exist in a latent space of lower dimensionality the input and/or the desired output. For example, the desired output may exist as multi-dimensional data including a time dimension, an amplitude value, and other measurements for each biological measurement. Thus, the output layer(s) may decode the latent biological representation from a lower (e.g., one or two dimension) representation into a higher representation output using learning parameters of the nodes of each layer. The layers may be fully connected or partially connected, or may be a single layer. Moreover, the layers can be arranged as convolutional layers, recurrent layers, attention layers and/or transformer layers or a combination thereof.

For example, an exemplary training methodology can be used to construct the parameters and/or hyperparameters of the DNN structure of the biological prediction model 530. An exemplary training methodology can include regularization and optimization.

Regularization, which encourages fewer significant connection weights and therefore better generalization, can include, e.g., batch normalization (e.g., each layer's outputs rescaled but preserve relative magnitudes), weight decay (e.g., each weight decreases a bit on each step such that inactive weights shrink away), L1 or L2 regularization (e.g., training cost prioritizes smaller weights or fewer weights), and dropout (e.g., weights are randomly zeroed out, preventing reliance on individual weights), among others or any combination thereof.

Optimization can include hyperparameter tuning, cross-validation, early stopping, speed of weight adjustments, among other techniques or any combination thereof.

Hyperparameter tuning can include building models, given the set of configurable structural and training parameters, using different combination of values to determine which combination results in the best final model (as quantified by the cross-validation performance metric below).

Cross-validation can include testing a combination of hyperparameter values by partitioning the training dataset into K subsets (“folds”), for each of which a model is trained using the remaining training data and the resulting model evaluated on the fold data to produce a performance metric; the total performance of this hyperparameter combo is quantified as the aggregate (sum or mean) of the fold performances, thus giving each training sample an equal contribution to the metric

Early stopping can include opting to stop training the model soon after the loss (error) on the validation dataset stop improving (in order to avoid overfitting); then rewind to recent best model in terms of validation loss. An early stopping delta parameter can include, e.g., a minimum amount of loss decrease that is considered an improvement. A patience parameter can define how many training epochs of no improvement to wait before stopping training.

Speed of weight adjustments can include one or more learning rate parameters. For example, an “Adam” algorithm makes adaptive adjustments to learning rate in order to balance training speed with stability of convergence using a learning rate parameter and/or custom learning rate scheduling. The learning rate parameter can adjust to initialize Adam algorithm, while a custom learning rate scheduling can varies the learning rate epoch after an epoch.

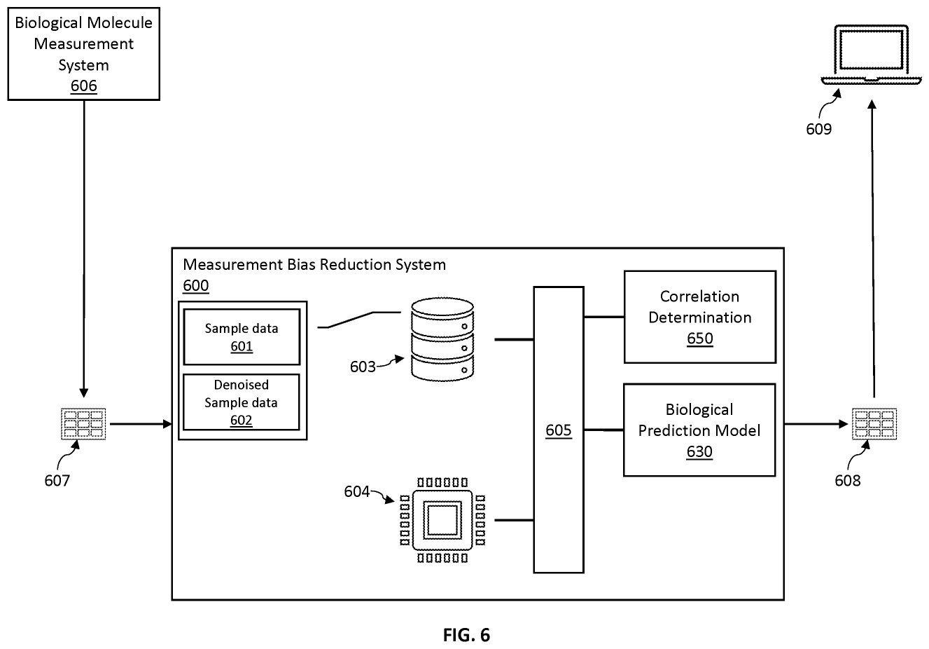

Referring now to FIG. 6, a block diagram is depicted illustrating a system for reducing noise in biological molecule measurement systems in accordance with one or more embodiments of the present disclosure.

In some embodiments, a measurement noise reduction system 600 may curate, collect, obtain, access or otherwise use sample data 601 from biological measurements 607 collected by a biological molecule measurement system 606, such as a proteomics, genomics, or other omics measurement and/or analysis system. The sample data 601 may be stored in a database 603 and processed to produce denoised sample data 602.

In some embodiments, the measurement noise reduction system 600 may include hardware components such as a processor(s) 604, which may include local or remote processing components. In some embodiments, the processor(s) 604 may include any type of data processing capacity, such as a hardware logic circuit, for example an application specific integrated circuit (ASIC) and a programmable logic, or such as a computing device, for example, a microcomputer or microcontroller that include a programmable microprocessor. In some embodiments, the processor(s) 604 may include data-processing capacity provided by the microprocessor. In some embodiments, the microprocessor may include memory, processing, interface resources, controllers, and counters. In some embodiments, the microprocessor may also include one or more programs stored in memory.

Similarly, the measurement noise reduction system 600 may include storage 603, such as one or more local and/or remote data storage solutions such as, e.g., local hard-drive, solid-state drive, flash drive, database or other local data storage solutions or any combination thereof, and/or remote data storage solutions such as a server, mainframe, database or cloud services, distributed database or other suitable data storage solutions or any combination thereof. In some embodiments, the storage 603 may include, e.g., a suitable non-transient computer readable medium such as, e.g., random access memory (RAM), read only memory (ROM), one or more buffers and/or caches, among other memory devices or any combination thereof.

In some embodiments, the measurement noise reduction system 600 may implement computer engines for correlation determination 650, a measurement prediction model 630, and/or noise correction 640. In some embodiments, the terms “computer engine” and “engine” identify at least one software component and/or a combination of at least one software component and at least one hardware component which are designed/programmed/configured to manage/control other software and/or hardware components (such as the libraries, software development kits (SDKs), objects, etc.).

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Examples of software may include software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

In some embodiments, to determine covariate molecules and/or measurements, the measurement noise reduction system 600 may include computer engines for the correlation determination 650, such as for the correlation determination 250/350 detailed above. In some embodiments, the computer engines for the correlation determination 650 may include dedicated and/or shared software components, hardware components, or a combination thereof. For example, the computer engines for the correlation determination 650 may include a dedicated processor and storage. However, in some embodiments, the computer engines for the correlation determination 650 may share hardware resources, including the processor(s) 604 and storage 603 of the measurement noise reduction system 600 via, e.g., a bus 605.

In some embodiments, to determine expected or predicted measurements, the measurement noise reduction system 600 may include computer engines for the measurement prediction model 630, such as for the measurement prediction model 130, 230 and/or 430 detailed above. In some embodiments, the computer engines for the correlation determination 650 may include dedicated and/or shared software components, hardware components, or a combination thereof. For example, the computer engines for the correlation determination 650 may include a dedicated processor and storage. However, in some embodiments, the computer engines for the correlation determination 650 may share hardware resources, including the processor(s) 604 and storage 603 of the measurement noise reduction system 600 via, e.g., a bus 605.

In some embodiments, to use expected or predicted measurements for noise reduction, the measurement noise reduction system 600 may include computer engines for the noise correction 640, such as for the noise correction 140 detailed above. In some embodiments, the computer engines for the correlation determination 650 may include dedicated and/or shared software components, hardware components, or a combination thereof. For example, the computer engines for the correlation determination 650 may include a dedicated processor and storage. However, in some embodiments, the computer engines for the correlation determination 650 may share hardware resources, including the processor(s) 604 and storage 603 of the measurement noise reduction system 600 via, e.g., a bus 605.

In some embodiments, the measurement noise reduction system 600 may output denoised biological measurements 608 for the biological measurements 607. The denoised biological measurements 608 may be output to a computing system 609 associated with a clinical or other user that associated with the biological measurements 607.

In some embodiments, the user may be a clinician utilizing clinical decision support software of the computing system 609. In some embodiments, clinical decision support software (CDSS) may be a health information technology that provides clinicians, staff, patients, and other individuals with knowledge and person-specific information to help health and health care to enhance decision-making in the clinical workflow. The CDSS may include computerized alerts and reminders to care providers and patients, clinical guidelines, condition-specific order sets, focused patient data reports and summaries, documentation templates, diagnostic support, and contextually relevant reference information, among other tools.

In some embodiments, the CDSS of the computing system 609 may include a knowledge-based system, including, without limitation a knowledge base of protein measurements and/or proteomic data, an inference engine, and a mechanism to communicate. The knowledge base may include the rules and associations of protein measurements and/or proteomic data, including, without limitation, IF-THEN rules. The inference engine may combine the rules from the knowledge base with the denoised biological measurements 608 to produce clinical support recommendations, such as recommended diagnoses, triage recommendations, study design recommendations, among other clinical support tasks or any combination thereof.

In some embodiments, the CDSS may include non-knowledge-based CDSS which may not use a knowledge base, but may instead use machine learning or other artificial intelligence. Machine learning (ML) based CDSS may be used to generate similar clinical decision support, but since systems based on ML may not explain the reasons for their conclusions, the ML may instead be employed as post-diagnostic systems, for suggesting patterns for clinicians to look into in more depth.

In some embodiments, the ML-based CDSS may include one or more AI and/or ML techniques, including those detailed further above, such as support-vector machines, artificial neural networks and genetic algorithms, among others or any combination thereof. For example, genetic algorithms, which are based on simplified evolutionary processes using directed selection to achieve optimal CDSS results, may use the selection algorithms to evaluate components of random sets of solutions to a problem based on the denoised biological measurements 608. The solutions that come out on top may then be recombined and mutated and run through the process again until the proper solution is discovered.

In some embodiments, the computing system 609 may include software and/or systems for regulatory-compliant reporting. In some embodiments, the computing system 609 may update electronic health records (EHR), insurance claims, drug study data, health study data, among other data records, with the denoised biological measurements 608. In some embodiments, the computing system 609 may include both the original biological measurements 607 as well as the denoised biological measurements 608 for auditability. As a result, the computing system 609 may be used to report data, including the denoised biological measurements 608, compliant with FDA, CLIA, or other regulatory standard or any combination thereof.

It is understood that at least one aspect/functionality of various embodiments described herein can be performed in real-time and/or dynamically. As used herein, the term “real-time” is directed to an event/action that can occur instantaneously or almost instantaneously in time when another event/action has occurred. For example, the “real-time processing,” “real-time computation,” and “real-time execution” all pertain to the performance of a computation during the actual time that the related physical process (e.g., a user interacting with an application on a mobile device) occurs, in order that results of the computation can be used in guiding the physical process.

As used herein, the term “dynamically” and term “automatically,” and their logical and/or linguistic relatives and/or derivatives, mean that certain events and/or actions can be triggered and/or occur without any human intervention. In some embodiments, events and/or actions in accordance with the present disclosure can be in real-time and/or based on a predetermined periodicity of at least one of: nanosecond, several nanoseconds, millisecond, several milliseconds, second, several seconds, minute, several minutes, hourly, several hours, daily, several days, weekly, monthly, etc.

In some embodiments, exemplary inventive, specially programmed computing systems and platforms with associated devices are configured to operate in the distributed network environment, communicating with one another over one or more suitable data communication networks (e.g., the Internet, satellite, etc.) and utilizing one or more suitable data communication protocols/modes such as, without limitation, IPX/SPX, X.25, AX.25, AppleTalk™, TCP/IP (e.g., HTTP), near-field wireless communication (NFC), RFID, Narrow Band Internet of Things (NBIOT), 3G, 4G, 5G, GSM, GPRS, WiFi, WiMax, CDMA, satellite, ZigBee, and other suitable communication modes.

The material disclosed herein may be implemented in software or firmware or a combination of them or as instructions stored on a machine-readable medium, which may be read and executed by one or more processors. A machine-readable medium may include any medium and/or mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others.

As used herein, the terms “computer engine” and “engine” identify at least one software component and/or a combination of at least one software component and at least one hardware component which are designed/programmed/configured to manage/control other software and/or hardware components (such as the libraries, software development kits (SDKs), objects, etc.).

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Computer-related systems, computer systems, and systems, as used herein, include any combination of hardware and software. Examples of software may include software components, programs, applications, operating system software, middleware, firmware, software modules, routines, subroutines, functions, methods, procedures, software interfaces, application program interfaces (API), instruction sets, computer code, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

In some embodiments, one or more of illustrative computer-based systems or platforms of the present disclosure may include or be incorporated, partially or entirely into at least one personal computer (PC), laptop computer, ultra-laptop computer, tablet, touch pad, portable computer, handheld computer, palmtop computer, personal digital assistant (PDA), cellular telephone, combination cellular telephone/PDA, television, smart device (e.g., smart phone, smart tablet or smart television), mobile internet device (MID), messaging device, data communication device, and so forth.

As used herein, term “server” should be understood to refer to a service point which provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Cloud servers are examples.

In some embodiments, as detailed herein, one or more of the computer-based systems of the present disclosure may obtain, manipulate, transfer, store, transform, generate, and/or output any digital object and/or data unit (e.g., from inside and/or outside of a particular application) that can be in any suitable form such as, without limitation, a file, a contact, a task, an email, a message, a map, an entire application (e.g., a calculator), data points, and other suitable data. In some embodiments, as detailed herein, one or more of the computer-based systems of the present disclosure may be implemented across one or more of various computer platforms such as, but not limited to: (1) FreeBSD, NetBSD, OpenBSD; (2) Linux; (3) Microsoft Windows™; (4) Open VMS™; (5) OS X (MacOS™); (6) UNIX™; (7) Android; (8) iOS™; (9) Embedded Linux; (10) Tizen™; (11) WebOS™; (12) Adobe AIR™; (13) Binary Runtime Environment for Wireless (BREW™); (14) Cocoa™ (API); (15) Cocoa™ Touch; (16) Java™ Platforms; (17) JavaFX™; (18) QNX™; (19) Mono; (20) Google Blink; (21) Apple WebKit; (22) Mozilla Gecko™; (23) Mozilla XUL; (24) .NET Framework; (25) Silverlight™; (26) Open Web Platform; (27) Oracle Database; (28) Qt™; (29) SAP NetWeaver™; (30) Smartface™; (31) Vexi™; (32) Kubernetes™ and (33) Windows Runtime (WinRT™) or other suitable computer platforms or any combination thereof. In some embodiments, illustrative computer-based systems or platforms of the present disclosure may be configured to utilize hardwired circuitry that may be used in place of or in combination with software instructions to implement features consistent with principles of the disclosure. Thus, implementations consistent with principles of the disclosure are not limited to any specific combination of hardware circuitry and software. For example, various embodiments may be embodied in many different ways as a software component such as, without limitation, a stand-alone software package, a combination of software packages, or it may be a software package incorporated as a “tool” in a larger software product.

For example, exemplary software specifically programmed in accordance with one or more principles of the present disclosure may be downloadable from a network, for example, a website, as a stand-alone product or as an add-in package for installation in an existing software application. For example, exemplary software specifically programmed in accordance with one or more principles of the present disclosure may also be available as a client-server software application, or as a web-enabled software application. For example, exemplary software specifically programmed in accordance with one or more principles of the present disclosure may also be embodied as a software package installed on a hardware device.

In some embodiments, illustrative computer-based systems or platforms of the present disclosure may be configured to handle numerous concurrent users that may be, but is not limited to, at least 100 (e.g., but not limited to, 100-999), at least 1,000 (e.g., but not limited to, 1,000-9,999), at least 10,000 (e.g., but not limited to, 10,000-99,999) , at least 100,000 (e.g., but not limited to, 100,000-999,999), at least 1,000,000 (e.g., but not limited to, 1,000,000-9,999,999), at least 10,000,000 (e.g., but not limited to, 10,000,000-99,999,999), at least 100,000,000 (e.g., but not limited to, 100,000,000-999,999,999), at least 1,000,000,000 (e.g., but not limited to, 1,000,000,000-999,999,999,999), and so on.

In some embodiments, illustrative computer-based systems or platforms of the present disclosure may be configured to output to distinct, specifically programmed graphical user interface implementations of the present disclosure (e.g., a desktop, a web app., etc.). In various implementations of the present disclosure, a final output may be displayed on a displaying screen which may be, without limitation, a screen of a computer, a screen of a mobile device, or the like. In various implementations, the display may be a holographic display. In various implementations, the display may be a transparent surface that may receive a visual projection. Such projections may convey various forms of information, images, or objects. For example, such projections may be a visual overlay for a mobile augmented reality (MAR) application.

As used herein, terms “cloud,” “Internet cloud,” “cloud computing,” “cloud architecture,” and similar terms correspond to at least one of the following: (1) a large number of computers connected through a real-time communication network (e.g., Internet); (2) providing the ability to run a program or application on many connected computers (e.g., physical machines, virtual machines (VMs)) at the same time; (3) network-based services, which appear to be provided by real server hardware, and are in fact served up by virtual hardware (e.g., virtual servers), simulated by software running on one or more real machines (e.g., allowing to be moved around and scaled up (or down) on the fly without affecting the end user).

In some embodiments, the illustrative computer-based systems or platforms of the present disclosure may be configured to securely store and/or transmit data by utilizing one or more of encryption techniques (e.g., private/public key pair, Triple Data Encryption Standard (3DES), block cipher algorithms (e.g., IDEA, RC2, RC5, CAST and Skipjack), cryptographic hash algorithms (e.g., MD5, RIPEMD-160, RTRO, SHA-1, SHA-2, Tiger (TTH), WHIRLPOOL, RNGs).

As used herein, the term “user” shall have a meaning of at least one user. In some embodiments, the terms “user”, “subscriber” “consumer” or “customer” should be understood to refer to a user of an application or applications as described herein and/or a consumer of data supplied by a data provider. By way of example, and not limitation, the terms “user” or “subscriber” can refer to a person who receives data provided by the data or service provider over the Internet in a browser session, or can refer to an automated software application which receives the data and stores or processes the data.

The aforementioned examples are, of course, illustrative and not restrictive.

Clause 1. A method comprising: obtaining, by the at least one processor, training biological measurement data from at least one multi-omics data source, the training biological measurement data comprising a plurality of training biological measurements for a plurality of biological measurements of a plurality of training subjects; determining, by the at least one processor, for a particular biological measurement of the plurality of biological measurements in the training biological measurement data, cross-biological measurement correlations between: at least one particular training biological measurement of the particular biological measurement and each covariate biological measurement of a plurality of covariate biological measurements associated with each covariate biological measurement, each covariate biological measurement being associated with a different measurement type relative to the training biological measurement data; updating, by the at least one processor, training for a biological measurement noise model to output at least one denoised biological measurement value based at least in part on cross-biological measurement correlations for the particular biological measurement, wherein the training comprises: inputting each covariate biological measurement associated with each covariate biological measurement; outputting the at least one de-noised biological measurement value; and updating the biological measurement noise model based at least in part on an error between the at least one de-noised biological measurement value and the at least one particular training biological measurement of the particular biological measurement; utilizing, by the at least one processor, the biological measurement noise model to predict new de-noised biological measurement values for the particular biological measurement in new samples based at least in part on each new covariate biological measurement associated with each new covariate biological measurement of the plurality of covariate biological measurements in the new samples; and correcting, by the at least one processor, the particular biological measurement data for the new samples based at least in part on the at least one de-noised biological measurement value.

Clause 2. The method of clause 1, wherein the at least one multi-omics data source comprises at least one proteomics measurement method.

Clause 3. The method of clause 2, wherein the at least one proteomics measurement method comprises at least one of: at least one immunoassay, at least one mass spectrometry.

Clause 4. The method of clause 1, wherein the particular biological measurement comprises a protein abundance measurement.

Clause 5. The method of clause 1, further comprising clinical decision support software configured to: determine, upon correcting the noise, at least one diagnosis recommendation based at least in part on the new particular biological measurement values.

Clause 6. The method of clause 5, wherein the clinical decision support software is further configured to: enter the at least one diagnosis recommendation into an electronic health record of a patient associated with the new samples.

Clause 7. The method of clause 5, wherein the clinical decision support software is further configured to: render, on a display of a computing device associated with a clinician, at least one notification representing the at least one diagnosis recommendation.

Clause 8. A method comprising: obtaining, by at least one processor, protein data from at least one sample, the protein data comprising a plurality of protein measurement values associated with a plurality of proteins in the at least one sample; obtaining, by the at least one processor, covariate protein data, the covariate protein data comprising a plurality of covariate protein measurement values associated with a plurality of covariate proteins; utilizing, by the at least one processor, a protein noise model to output at least one de-noised protein measurement value for a particular protein of the plurality of proteins based at least in part on cross-protein correlations between: at least one particular training protein measurement of the particular protein and each covariate protein measurement of the plurality of covariate proteins; correcting, by the at least one processor, the protein data for the at least one sample based at least in part on the at least one de-noised protein measurement value.

Clause 9. The method of clause 8, wherein the at least one multi-omics data source comprises at least one proteomics measurement method.

Clause 10. The method of clause 9, wherein the at least one proteomics measurement method comprises at least one of: at least one immunoassay, and at least one mass spectrometry.

Clause 11. The method of clause 8, wherein the particular biological measurement comprises a protein abundance measurement.

Clause 12. The method of clause 8, further comprising clinical decision support software configured to: determine, upon correcting the noise, at least one diagnosis recommendation based at least in part on the new particular biological measurement values.

Clause 13. The method of clause 12, wherein the clinical decision support software is further configured to: enter the at least one diagnosis recommendation into an electronic health record of a patient associated with the new samples.

Clause 14. The method of clause 12, wherein the clinical decision support software is further configured to: render, on a display of a computing device associated with a clinician, at least one notification representing the at least one diagnosis recommendation.

While one or more embodiments of the present disclosure have been described, it is understood that these embodiments are illustrative only, and not restrictive, and that many modifications may become apparent to those of ordinary skill in the art, including that various embodiments of the inventive methodologies, the illustrative systems and platforms, and the illustrative devices described herein can be utilized in any combination with each other. Further still, the various steps may be carried out in any desired order (and any desired steps may be added and/or any desired steps may be eliminated).

What is claimed is:

1. A computer-implemented method comprising:
obtaining health-related data for a subject, the health-related data being organized into a plurality of semantically meaningful input groups;
processing each input group through a respective group-specific transformation pipeline relative to a common internal semantic basis to generate a corresponding group representation comprising a group-specific deviation from the common internal semantic basis;
determining, for each input group, a respective adaptive weighting factor for scaling a contribution of a corresponding group-specific deviation in a common slot-based semantic space defined by the common internal semantic basis;
fusing, within the common slot-based semantic space, the group-specific deviations according to the respective adaptive weighting factors with the common internal semantic basis to generate an integrated representation in which the plurality of semantically meaningful input groups are aligned along common conceptual axes;
inputting the integrated representation into a machine-learning model to generate a latent biological representation of a biological state of the subject; and
using the latent biological representation to generate at least one reconstruction output and at least one health-related prediction.

2. The method of claim 1, wherein the plurality of semantically meaningful input groups comprises at least a proteomics group, a clinical covariates group, and a metabolomics group.

3. The method of claim 1, wherein the common internal semantic basis comprises a plurality of learned shared concept anchors defining the common slot-based semantic space.

4. The method of claim 1, wherein processing each input group through the respective group-specific transformation pipeline comprises converting features of the input group into an internal token representation and refining the internal token representation using intra-group contextual modeling before said fusing.

5. The method of claim 1, wherein processing each input group through the respective group-specific transformation pipeline comprises interacting the input group with the common internal semantic basis through attention-based pooling to generate the corresponding group-specific deviation.

6. The method of claim 1, wherein the respective adaptive weighting factor for each input group comprises a learned gate representing at least one of a confidence factor, a salience factor, or an influence factor for the corresponding input group.

7. The method of claim 1, further comprising refining the integrated representation with a shared encoder before said inputting the integrated representation into the machine-learning model.

8. The method of claim 1, wherein the at least one reconstruction output comprises a denoised reconstruction generated by reconstructing entries intentionally corrupted or masked during training.

9. The method of claim 1, wherein the at least one health-related prediction comprises at least one of a clinical outcome prediction, a disease-state classification, a continuous phenotype estimate, a survival-related prediction, or a treatment-response prediction.

10. The method of claim 1, further comprising performing an ablation-based analysis by removing or attenuating one of the plurality of semantically meaningful input groups and measuring an effect on the at least one health-related prediction.

11. A system comprising:
at least one processor; and
a memory storing instructions that, when executed by the at least one processor, cause the system to:
obtain health-related data for a subject, the health-related data being organized into a plurality of semantically meaningful input groups;
process each input group through a respective group-specific transformation pipeline relative to a common internal semantic basis to generate a corresponding group representation comprising a group-specific deviation from the common internal semantic basis;
determine, for each input group, a respective adaptive weighting factor for scaling a contribution of a corresponding group-specific deviation in a common slot-based semantic space defined by the common internal semantic basis;
fuse, within the common slot-based semantic space, the group-specific deviations according to the respective adaptive weighting factors with the common internal semantic basis to generate an integrated representation in which the plurality of semantically meaningful input groups are aligned along common conceptual axes;
input the integrated representation into a machine-learning model to generate a latent biological representation of a biological state of the subject; and
use the latent biological representation to generate at least one reconstruction output and at least one health-related prediction.

12. The system of claim 11, wherein the plurality of semantically meaningful input groups comprises at least a proteomics group, a clinical covariates group, and a metabolomics group.

13. The system of claim 11, wherein the common internal semantic basis comprises a plurality of learned shared concept anchors defining the common slot-based semantic space.

14. The system of claim 11, wherein processing each input group through the respective group-specific transformation pipeline comprises converting features of the input group into an internal token representation and refining the internal token representation using intra-group contextual modeling before said fusing.

15. The system of claim 11, wherein processing each input group through the respective group-specific transformation pipeline comprises interacting the input group with the common internal semantic basis through attention-based pooling to generate the corresponding group-specific deviation.

16. The system of claim 11, wherein the respective adaptive weighting factor for each input group comprises a learned gate representing at least one of a confidence factor, a salience factor, or an influence factor for the corresponding input group.

17. The system of claim 11, wherein the instructions, when executed by the at least one processor, further cause the system to refine the integrated representation with a shared encoder before said inputting the integrated representation into the machine-learning model.

18. The system of claim 11, wherein the at least one reconstruction output comprises a denoised reconstruction generated by reconstructing entries intentionally corrupted or masked during training.

19. The system of claim 11, wherein the at least one health-related prediction comprises at least one of a clinical outcome prediction, a disease-state classification, a continuous phenotype estimate, a survival-related prediction, or a treatment-response prediction.

20. The system of claim 11, wherein the instructions, when executed by the at least one processor, further cause the system to perform an ablation-based analysis by removing or attenuating one of the plurality of semantically meaningful input groups and measuring an effect on the at least one health-related prediction.
